(19) United States
(12) Patent Application Publication
(10) Pub. No.: US 2026/0158278 A1
(43) Pub. Date: Jun. 11, 2026

(54) PREDICTION OF DBS CLINICAL EFFECTS

(71) Applicant: Boston Scientific Neuromodulation Corporation, Valencia, CA (US)

(72) Inventor: Leon Mauricio Juarez Paz, Berlin (DE)

(21) Appl. No.: 19/395,823

(22) Filed: Nov. 20, 2025

Related U.S. Application Data
(60) Provisional application No. 63/729,793, filed on Dec. 9, 2024.

(51) Int. Cl. A61N 1/36

(52) U.S. Cl. CPC A61N 1/3615; A61N 1/36171; A61N 1/36175; A61N 1/36182

(57) ABSTRACT

A system may include a neurostimulator including electrodes and a processing system configured to perform a process. The process may include receiving lead placement information and clinical history for the patient. The clinical history may include both first clinical effect data corresponding to times when the patient is not being treated with a therapy and second clinical effect data corresponding to times when the patient is being treated with the therapy. The process may include employing at least one trained machine-learning model to provide predicted clinical effects, corresponding to untested stimulation parameter sets for the neurostimulator, based on the lead placement information, the first clinical effect data and the second clinical effect data. A parameter set may be identified for use in delivering a therapy based at least in part on the predicted clinical effects for the untested stimulation parameter sets.

### CLAIM OF PRIORITY

This application claims the benefit of U.S. Provisional Application No. 63/729,793, filed on Dec. 9, 2024, which is hereby incorporated by reference in its entirety.

### TECHNICAL FIELD

This document relates generally to medical devices, and more particularly, to systems, devices and methods for determining parameter sets for a neurostimulation therapy.

### BACKGROUND

Medical devices may include therapy-delivery devices configured to deliver a therapy to a patient and/or monitors configured to monitor a patient condition via user input and/or sensor(s). Examples include wearable devices such as but not limited to, transcutaneous electrical neural stimulators (TENS), external or implantable stimulation devices such as but not limited to spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, and the like.

A therapy device may be configured or programmed to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. The description that follows will generally focus on the use of the invention within a DBS context. DBS has been applied therapeutically for the treatment of neurological disorders, including PD, essential tremor, dystonia, and epilepsy, to name but a few.

Neurostimulation systems, whether implantable or external, typically includes one or more electrode-carrying stimulation leads, which are implanted at the desired stimulation site, and a neurostimulator, used externally or implanted remotely from the stimulation site, but coupled either directly to the neurostimulation lead(s) or indirectly to the neurostimulation lead(s) via a lead extension. The neurostimulation system may further comprise an external control device configured to instruct the neurostimulator to generate electrical stimulation pulses in accordance with selected stimulation parameters. Typically, the stimulation parameters programmed into the neurostimulator can be adjusted by manipulating controls on the external control device to modify the electrical stimulation provided by the neurostimulator system to the patient.

Non-optimal electrode placement and stimulation parameter selections may result in excessive energy consumption due to stimulation that is set at too high amplitude, too wide a pulse duration, or too fast a frequency; inadequate or marginalized treatment due to stimulation that is set at too low an amplitude, too narrow a pulse duration, or too slow a frequency; or stimulation of neighboring cell populations that may result in undesirable side effects.

The large number of electrodes available, combined with the ability to generate a variety of complex stimulation pulses, presents a huge selection of stimulation parameter sets to the clinician or patient. In the context of DBS, neurostimulation leads with a complex arrangement of electrodes that not only are distributed axially along the leads, but are also distributed circumferentially around the neurostimulation leads as segmented electrodes, can be used. To facilitate such selection, the clinician generally programs the external control device, and if applicable the neurostimulator, through a computerized programming system. This programming system can be a self-contained hardware/software system, or can be defined predominantly by software running on a standard personal computer (PC) or mobile platform. The PC or custom hardware may actively control the characteristics of the electrical stimulation generated by the neurostimulator to allow the optimum stimulation parameters to be determined based on patient feedback and to subsequently program the external control device with the optimum stimulation parameters. The computerized programming system may be used to instruct the neurostimulator to apply electrical stimulation to test placement of the leads and/or electrodes, thereby assuring that the leads and/or electrodes are implanted in effective locations within the patient. The system may also instruct the user how to improve the positioning of the leads, or confirm when a lead is well-positioned. Once the leads are correctly positioned, a fitting procedure, which may be referred to as a navigation session, may be performed using the computerized programming system to program the external control device, and if applicable the neurostimulator, with a set of stimulation parameters that addresses the neurological disorder(s). There is a need for methods and systems that assist a clinician in determining optimum stimulation parameters for treating the patient.

### SUMMARY

An example (e.g., “Example 1”) of a system may include a neurostimulator including electrodes and a processing system configured to perform a process. The process may include receiving lead placement information for the patient, where placement information may be indicative of where at least one lead for the neurostimulator is implanted in the patient. The process may include receiving clinical history for the patient, where the clinical history may include both first clinical effect data corresponding to times when the patient is not being treated with a therapy and second clinical effect data corresponding to times when the patient is being treated with the therapy. The process may include employing at least one trained machine-learning model to provide predicted clinical effects, corresponding to untested stimulation parameter sets for the neurostimulator, based on the lead placement information, the first clinical effect data and the second clinical effect data. A parameter set may be identified for use in delivering a therapy based at least in part on the predicted clinical effects for the untested stimulation parameter sets.

In Example 2, the subject matter of Example 1 may optionally be configured such that the parameter set is identified using the predicted clinical effects to seed a search algorithm and implementing the search algorithm to determine the parameter set.

In Example 3, the subject matter of any one or more of Examples 1-2 may optionally be configured such that the parameter set is identified using the predicted clinical effects to determine when to end searching for the parameter set.

In Example 4, the subject matter of any one or more of Examples 1-3 may optionally be configured such that the parameter set is identified by the at trained machine-learning model(s) based on the lead placement information, the first clinical effect data and the second clinical effect data.

In Example 5, the subject matter of any one or more of Examples 1-4 may optionally be configured such that the machine-learning model(s) is (are) configured to suggest at least one of a lead entry point, a lead trajectory, a lead orientation, or a neural target based on the predicted clinical effects.

In Example 6, the subject matter of any one or more of Examples 1-5 may optionally be configured such that the trained machine-learning model(s) is (are) configured to suggest, based on the predicted clinical effects, whether the patient is likely a responder or a non-responder to therapy.

In Example 7, the subject matter of any one or more of Examples 1-6 may optionally be configured such that the trained machine-learning model(s) includes (include) a regression model.

An example (e.g., “Example 8”) of a system may include one or more processors and one or more memory devices storing instruction, which when executed by the processor(s) cause the processor(s) to perform operations. The operations may include accessing data stored in one or databases where the data includes first clinical effect data corresponding to a first set of patient states for times when therapy is not delivered, second clinical effect data corresponding to a second set of patient states for times when therapy is delivered, and lead placement information for a neurostimulator that has a plurality of electrodes and that is configured to use at least one stimulation parameter set to deliver stimulation through at least some of the plurality of electrodes. The lead placement information may be indicative of where at least one lead is implanted in the patient. The operations may include providing machine learning to train at least one machine-learning model to predict a clinical effect for the neurostimulator programmed with untested stimulation parameter sets based on the lead placement information, the first clinical effect data, and the second clinical effect data.

In Example 9, the subject matter of Example 8 may optionally be configured such that the data includes a plurality of tested stimulation sets and corresponding third clinical effect data. Each of the tested stimulation sets may be used to deliver electrical stimulation through at least one lead implanted with a lead placement, and a corresponding instance of the third clinical effect data is determined when the electrical stimulation is delivered. The machine-learning model(s) may be configured to predict the clinical effect based further on the third clinical effect data and the tested stimulation sets.

In Example 10, the subject matter of Example 9 may optionally be configured such that the tested stimulation sets include at least one amplitude, at least one frequency and at least one pulse width for the electrical stimulation, active electrodes and electrode fractionalization(s) for distributing the electrical stimulation among the active electrodes. The machine learning model(s) may be configured to predict a clinical effect based further on the amplitude(s), the frequency(ies), the pulse width(s), the active electrodes and/or the electrode fractionalization(s).

In Example 11, the subject matter of any one or more of Examples 8-10 may optionally be configured such that the machine-learning model(s) includes (include) a regression model.

In Example 12, the subject matter of any one or more of Examples 8-11 may optionally be configured such that the lead placement information includes an implanted lead position.

In Example 13, the subject matter of Example 12 may optionally be configured such that the lead placement information includes information regarding at least one of a lead entry point, a lead trajectory, a lead orientation, or a neural target.

In Example 14, the subject matter of any one or more of Examples 12-13 may optionally be configured such that the lead placement information includes relative lead position with respect to neuroanatomy or anatomical landmarks or anatomical coordinates defined by an anatomy of the patient or a stereotactic frame.

In Example 15, the subject matter of any one or more of Examples 12-14 may optionally be configured such that the lead placement information includes information regarding an identifier of a medical facility or medical provider that that performed a lead implant procedure.

Example 16 includes subject matter (such as a method, means for performing acts, machine readable medium including instructions that when performed by a machine cause the machine to perform acts, or an apparatus to perform). The subject matter may use a processing system to perform a process that includes: receiving lead placement information for a patient where the lead placement information may be indicative of where lead(s) for a neurostimulator is (are) implanted in the patient; receiving clinical history for the patient where the clinical history may include both first clinical effect data corresponding to times when the patient is not being treated with a therapy and second clinical effect data corresponding to times when the patient is being treated with the therapy; employing trained machine-learning model(s) to provide predicted clinical effects, corresponding to untested stimulation parameter sets for the neurostimulator, based on the lead placement information, the first clinical effect data and the second clinical effect data; and identifying a parameter set for use in delivering a therapy based at least in part on the predicted clinical effects for the untested stimulation parameter sets.

In Example 17, the subject matter of Example 16 may optionally be configured such that the parameter set is identified using the predicted clinical effects to seed a search algorithm and implementing the search algorithm to determine the parameter set.

In Example 18, the subject matter of any one or more of Examples 16-17 may optionally be configured such that the parameter set is identified using the predicted clinical effects to determine when to end searching for the parameter set.

In Example 19, the subject matter of any one or more of Examples 16-18 may optionally be configured such that the parameter set is identified by the trained machine-learning model(s) based on the lead placement information, the first clinical effect data and the second clinical effect data.

In Example 20, the subject matter of any one or more of Examples 16-19 may optionally be configured such that the trained machine-learning model(s) is (are) used to suggest a lead entry point, a lead trajectory, a lead orientation, and/or a neural target based on the predicted clinical effects.

In Example 21, the subject matter of any one or more of Examples 16-20 may optionally be configured such that the trained machine-learning model(s) is (are) used to suggest, based on the predicted clinical effects, whether the patient is likely a responder or a non-responder to therapy.

In Example 22, the subject matter of any one or more of Examples 16-21 may optionally be configured such that the trained machine-learning model(s) includes (include) a regression model.

Example 23 includes subject matter (such as a method, means for performing acts, machine readable medium including instructions that when performed by a machine cause the machine to perform acts, or an apparatus to perform). The subject matter may use a processing system to perform a process that includes accessing data stored in one or databases. The data may include first clinical effect data corresponding to a first set of patient states for times when therapy is not delivered, second clinical effect data corresponding to a second set of patient states for times when therapy is delivered, and lead placement information for a neurostimulator that has a plurality of electrodes and that is configured to use at least one stimulation parameter set to deliver stimulation through at least some of the plurality of electrodes. The lead placement information may be indicative of where at least one lead is implanted in the patient. The process may include providing machine learning to train machine-learning model(s) to predict a clinical effect for the neurostimulator programmed with untested stimulation parameter sets based on the lead placement information, the first clinical effect data, and the second clinical effect data.

In Example 24, the subject matter of Example 23 may optionally be configured such that the data includes a plurality of tested stimulation sets and corresponding third clinical effect data, and each of the plurality of tested stimulation sets are used to deliver electrical stimulation through at least one lead implanted with a lead placement, and a corresponding instance of the third clinical effect data is determined when the electrical stimulation is delivered. The process may include predicting, using the at least one machine-learning model, the clinical effect based further on the third clinical effect data and the plurality of tested stimulation sets.

In Example 25, the subject matter of Example 24 may optionally be configured such that the tested stimulation sets include amplitude(s), frequency(ies) and pulse width(s) for the electrical stimulation, active electrodes, and electrode fractionalization(s) for distributing the electrical stimulation among the active electrodes. The process may include predicting, using the machine learning model(s), the clinical effect based further on the amplitude(s), the frequency(ies), the pulse width(s), the active electrodes and/or the electrode fractionalization(s).

In Example 26, the subject matter of any one or more of Examples 21-25 may optionally be configured such that the trained machine-learning model(s) includes (include) a regression model.

In Example 27, the subject matter of any one or more of Examples 21-26 may optionally be configured such that the lead placement information includes an implanted position of at least one deep brain stimulation (DBS) lead.

In Example 28, the subject matter of Example 27 may optionally be configured such that the lead placement information includes information regarding a lead entry point, a lead trajectory, a lead orientation, and/or a neural target.

In Example 29, the subject matter of any one or more of Examples 27-28 may optionally be configured such that the lead placement information includes relative lead position with respect to neuroanatomy or anatomical landmarks, anatomical coordinates defined by an anatomy of the patient, or a stereotactic frame.

In Example 30, the subject matter of any one or more of Examples 27-29 may optionally be configured such that the lead placement information includes information regarding an identifier of a medical facility or medical provider that implanted the at least one DBS lead.

Example 31 includes subject matter that includes non-transitory machine-readable medium including instructions, which when executed by a machine, cause the machine to perform a method. The method may include, by way of example and not limitation, any of the subject matter for one or more of Examples 16-30. The machine-readable medium may include instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine-readable medium” is intended to include at least one machine-readable medium (e.g., two or more media which may be of the same type of media (such as but not limited to different nonvolatile semiconductor memory arrays) or different type of media (such as but not limited to a hard disk and a non-volatile semiconductor memory array). Furthermore, the term “machine” may include at least one processor, including one processor to implement all of the instructions, at least two processors where one processor operates on some of the instructions and other processor(s) operate on other instructions, or at least two processors where each processor is capable of operating on the same instructions. Thus, for example, distributed systems or systems with shared resources are contemplated.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates an example of an electrical stimulation system that may be used to deliver deep brain stimulation (DBS).

FIG. 2 illustrates an example of an implantable pulse generator (IPG) that may be used in a DBS system.

FIGS. 3A-3B illustrate examples of leads that may be coupled to an IPG to deliver electrostimulation such as DBS.

FIG. 4 illustrates an example of a computing device for programming or controlling the operation of an electrostimulation system.

FIG. 5 illustrates, by way of example and not limitation, the electrical therapy-delivery system.

FIG. 6 illustrates, by way of example and not limitation, a stimulation parameter control system and a part of the environment in which it may operate.

FIG. 7 illustrates a therapy being delivered according to a parameter set.

FIG. 8 illustrates a therapy space, which includes different parameter sets potentially available for delivering the therapy.

FIG. 9 illustrates, by way of example and not limitation, a two-dimensional plot of location and amplitude for measured and estimated outcomes (e.g., clinical effects) and sensed and estimated data.

FIG. 10 illustrates, by way of example and not limitation, a stimulation effects map having multidimension relationships between a stimulation parameter set (illustrated as including N parameter(s)) and each of a patient response and sensed data.

FIG. 11 illustrates, by way of example and not limitation, available stimulation parameter sets.

FIG. 12 illustrates, by way of example and not limitation, a method for providing a stimulation effects map which may be used to choose a parameter set to be tested or used for therapy

FIG. 13 illustrates, by way of example and not limitation, a Graphical User Interface (GUI) operable on an external device where the GUI is capable of providing a clinician with a visual indication of how stimulation selected for a patient will interact with the tissue in which the electrodes are implanted.

FIG. 14 illustrates, by way of example and not limitation, an accumulated database with data collected from fitting sessions conducted for a plurality of patients (and, possibly, for multiple hemispheres of the patients).

FIG. 15 illustrates, by way of example and not limitation, an algorithm that can be used to predict effective therapeutic stimulation parameters (or stimulation parameters that may cause side effects) for a subject patient based on an accumulated database, as shown in FIG. 14.

FIG. 16 illustrates, by way of example and not limitation, a method that uses trained machine-learning model(s) (e.g., regression model) to seed a search space for a DBS optimization algorithm to find the desired parameter set used in a programmed therapy.

FIG. 17 illustrates, by way of example and not limitation, a method that uses trained machine-learning model(s) (e.g., regression model) to determine when to end the search that is being performed by a DBS optimization algorithm to find the desired parameter set used in a programmed therapy.

FIG. 18 illustrates, by way of example and not limitation, a method that uses trained machine-learning model(s) (e.g., regression model) to seed a search space for a DBS optimization algorithm to find the desired parameter set used in a programmed therapy and that uses trained machine-learning model(s) (e.g., regression model) to determine when to end the search that is being performed by a DBS optimization algorithm to find the desired parameter set used in a programmed therapy.

FIG. 19 illustrates, by way of example and not limitation, a method that uses trained machine-learning model(s) (e.g., regression model) to directly find the desired parameter set used in a programmed therapy.

FIG. 20 is a flowchart depicting machine-learning pipeline, according to some examples.

FIG. 21 illustrates, by way of example and not limitation, a process for creating machine-learning models that may be used to predict parameter sets from untested stimulation settings.

FIG. 22 illustrates, by way of example and not limitation, a process for using trained machine learning regression model(s) to predict clinical effects for untested stimulation settings.

FIGS. 23A-23C illustrate, by way of example and not limitation, imaging of a lead with respect to targeted tissue such as the STN.

FIGS. 24A-24D illustrate, by way of example and not limitation, training data sets used to train a model and test data sets for evaluating the model that provide a proof of concept for training a regression model using data from many patients to train the model to predict clinical effects based on lead location and clinical effect data with and without therapy, and using other data from patients to test the model.

FIGS. 25A-25B illustrate, by way of example and not limitation, a training set for a ring mode and a test set for a direction mode, respectively.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments described herein use databases with information about the location of DBS leads and electrodes in relation to stimulation targets and the clinical effects of previously tested stimulation settings from several patients to train regression models to predict the clinical effect of stimulation settings and lead locations. By way of example and not limitation, the trained regression models may be configured to provide neurosurgeons with information about implanting parameters such as a lead angle that could result in the best beneficial clinical outcomes that a particular patient may get from the DBS therapy. The trained regression models may be configured to improve the performance of algorithms that aim to automatically optimize stimulation settings. The trained regression models may be configured to help algorithms (e.g., DBS optimization algorithms) in the exploration of additional stimulation parameters like e.g., Pulse Width and Frequency. The trained regression models may be configured to help clinicians and algorithms determine the ceiling of the clinical beneficial effect of DBS.

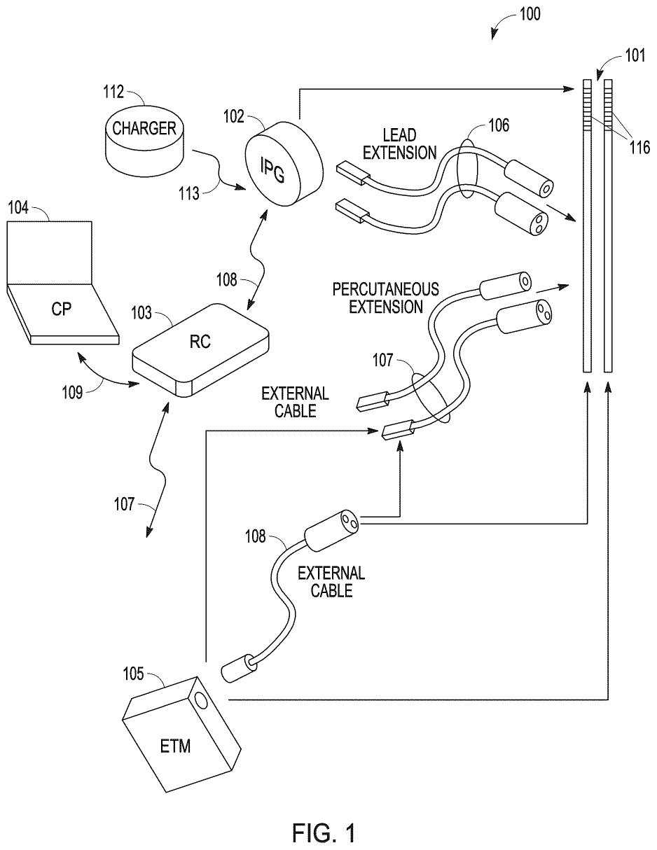

FIG. 1 illustrates, by way of example and not limitation, an electrical stimulation system 100, which may be used to deliver DBS. The electrical stimulation system 100 may generally include a one or more (illustrated as two) of implantable neurostimulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neurostimulation lead(s) 101, which carry a plurality of electrodes 116. The electrodes, when implanted in a patient, form an electrode arrangement. As illustrated, the neurostimulation leads 101 may be percutaneous leads with the electrodes arranged in-line along the neurostimulation leads or about a circumference of the neurostimulation leads. Any suitable number of neurostimulation leads can be provided. The IPG 102 includes pulse generation circuitry that delivers electrical stimulation energy in the form of a pulsed electrical waveform (i.e., a temporal series of electrical pulses) to the electrodes in accordance with a set of stimulation parameters.

The ETM 105 may also be physically connected via the percutaneous lead extensions 107 and external cable 108 to the neurostimulation lead(s) 101. The ETM 105 may have similar pulse generation circuitry as the IPG 102 to deliver electrical stimulation energy to the electrodes in accordance with a set of stimulation parameters. A programming process may be used to test different parameter sets. The ETM 105 is a non-implantable device that may be used on a trial basis after the neurostimulation leads 101 have been implanted and prior to implantation of the IPG 102, to test the responsiveness of the stimulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different stimulation parameter sets. The IPG 102 may also be operated to modify the programmed stimulation parameters to actively control the characteristics of the electrical stimulation energy output by the IPG 102. A clinician may use the CP 104 to program stimulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed stimulation parameters provided by the CP 104 may also be used to program the RC 103, so that the stimulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical stimulation generated by the IPG 102 to allow the desired parameters to be determined based on patient feedback or other feedback and for subsequently programming the IPG 102 with the desired stimulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the neurostimulation leads, and select and program the IPG with stimulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

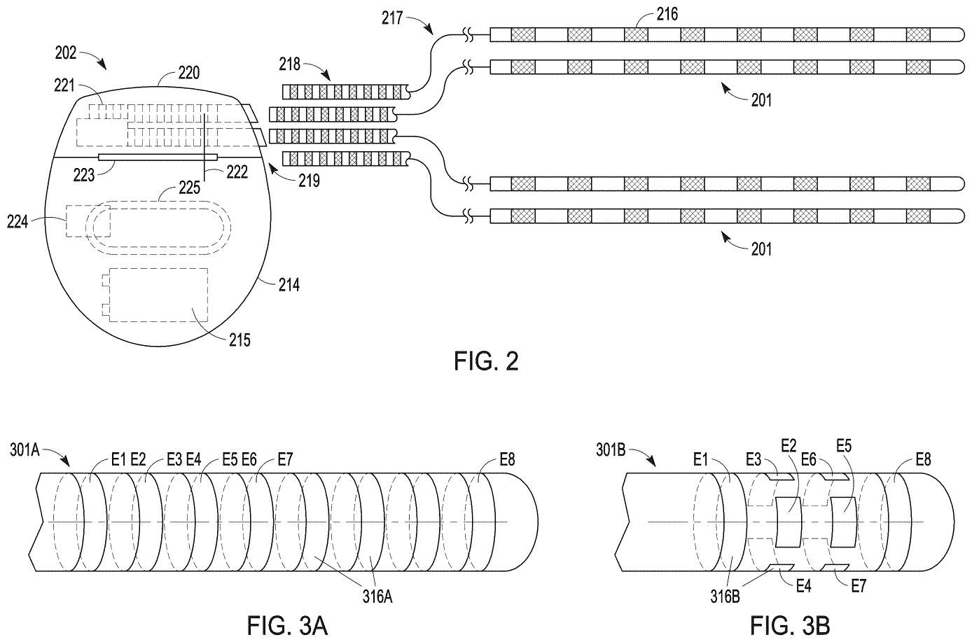

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be directional so that stimulation may be aligned with the target neurons after the neurons have been located such as based on the recorded signals. The electrodes 216 may include one or more ring electrodes, and/or one or more sets of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a Radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, MICS, and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 may also be implanted underneath the scalp closer to the location of the electrodes'implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes may either be used (an active electrode) or unused (OFF). When the electrode is used, the electrode may be used as an anode or cathode and carry anodic or cathodic current. The anodic energy contributions may be distributed across more than one anode and the cathodic energy contributions may be distributed across more than one cathode (e.g., electrode fractionalization). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead.

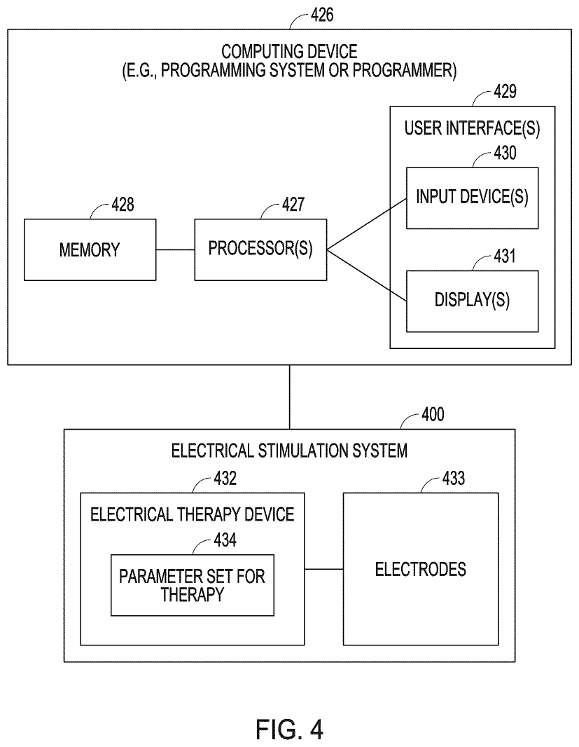

FIG. 4 illustrates, by way of example and not limitation, a computing device 426 for programming or controlling the operation of an electrical stimulation system 400. The computing device 426 may include a processor 427, a memory 428, and user interface(s) 429, which may include input device(s) 430 and display(s) 431. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1.

The computing device 426, also referred to as a programming system mor programmer, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. In some examples, the computing device 426 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, the closed-loop algorithm for determining or updating stimulation parameters may be implemented in a mobile device, such as a smartphone, which is connected to the IPG or an evaluating device (e.g., a wristband or watch). These devices may also record and send information to the clinician.

The processor 427 may include one or more processors that may be local to the user or non-local to the user or other components of the computing device 426. In an example, the processor 427 may execute instructions (e.g., stored in the memory 428) to determine a search space of electrode configurations and parameter values, and identify or update one or more stimulation settings that are selectable for use in electrostimulation therapies such as DBS. The search space may include a collection of available electrodes, possible electrode configurations, and possible values or value ranges of one or more stimulation parameters that may be applied to selected electrodes to deliver electrostimulation. The search space may be specific to a particular lead or a type of lead with respect to a specific neural target. As a result, for different leads or types of lead and/or for different neural targets, the processor 427 may determine respective different search spaces. A modulation configuration, which may be referred to as a stimulation setting, includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, and like.

The processor 427 may identify or modify a stimulation setting from the search space through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings, and/or instructions for implementing a testing process for testing stimulation parameters. The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

The input device(s) 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries. The display(s) 431 may be any suitable display or presentation device, such as a monitor, screen, display, or the like, and may include a printer. The display(s) 431 may be a part of a user interface configured to display information about stimulation settings (e.g., electrode configurations and stimulation parameter values and value ranges) and user control elements for programming a stimulation setting into an IPG.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

The electrical stimulation system 400 may include an electrical therapy device 432 configured to deliver an electrical therapy to electrodes 433 to treat a condition in accordance with a programmed parameter set 434 for the therapy. The illustrated electrical stimulation system 400 may be a DBS system. In some embodiments, the illustrated electrical stimulation system 400 may be an SCS system to treat pain using directional lead(s).

A modulation configuration, which may be referred to as a stimulation setting (e.g., parameter set), includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. By way of example and not limitation, the stimulation parameters may include current amplitude values, current fractionalization across electrodes, stimulation frequency, stimulation pulse width, and the like. Electrodes are capable of being selectively programmed to act as anodes (positive), cathodes (negative), or left off (zero). The electrical therapy device 432 may be configured to control or vary other parameters including, but are not limited to, the amplitude, pulse width, rate (or frequency), ON/OFF timing, and the like. Each electrode configuration, along with the electrical pulse parameters, can be referred to as a “neuromodulation parameter set.” Each set of neuromodulation parameters, including fractionalized current distribution to the electrodes (as percentage cathodic current, percentage anodic current, or off), may be stored and combined into a neuromodulation program that can then be used to modulate multiple regions within the patient.

The electrodes 433 may be distributed in an electrode arrangement using one or more leads. The electrical therapy device 432 may include a plurality of independent sources such as independent current sources for each electrode. The electrical therapy device 432 may be configured as a multi-channel (such as but not limited to four channels) system capable of simultaneously and independently generating and delivering separate stimulation waveforms to different electrode combinations. The channels may be referred to as timing channels.

Some embodiments of the electrical therapy device 432 may include electrical sensing circuitry configured to sense electrical activity (e.g., local field potentials, evoked compound actions potentials, evoked resonant neural activity (ERNA), electrospinogram, or other electrical signals) using at least some of the electrodes. Some embodiments of the neuromodulation device may include other sensor(s) that may be used to control the neuromodulation or provide context for the therapy or other events, or to detect events.

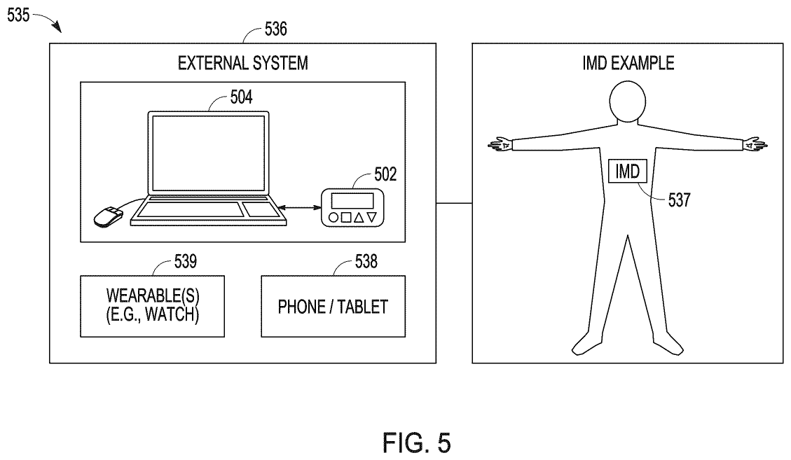

FIG. 5 illustrates, by way of example and not limitation, the electrical therapy-delivery system. The illustrated system 535 includes an external system 536 that may include at least one programming device or programming system such as computing device 426 in FIG. 4. The illustrated external system 536 may include a clinician programmer 504, similar to CP 104 in FIG. 1, configured for use by a clinician to communicate with and program the neuromodulator, and a remote control device 503, similar to RC 103 in FIG. 1, configured for use by the patient to communicate with and program the neuromodulator. For example, the remote control device 503 may allow the patient to turn a therapy on and off and/or may allow the patient to adjust patient-programmable parameter(s) of the plurality of stimulation parameters. FIG. 5 illustrates an IMD 537, although the therapy device may be an external device such as a wearable device. The external system 536 may include a network of computers, including computer(s) remotely located from the IMD 537 that are capable of communicating via one or more communication networks with the programmer 504 and/or the remote control device 503. The remotely located computer(s) and the IMD 537 may be configured to communicate with each other via another external device such as the programmer 504 or the remote control device 503. The remote control device 503 and/or the programmer 504 may allow a user (e.g., patient and/or clinician or rep) to answer questions as part of a data collection process. The external system 536 may include personal devices such as a phone or tablet 538, wearables such as a watch 539, sensors or therapy-applying devices. The watch may include sensor(s), such as sensor(s) for detecting activity, motion and/or posture. The phone and/or tablet may include camera(s), microphone(s), accelerometer(s) or other sensors that can be used to provide feedback. Other wearable sensor(s) may be configured for use to detect activity, motion and/or posture of the patient.

There may be a very large number of potential combinations of parameter values for a parameter set. Optimization algorithms may be used to enable machine learning models to learn from data. For example, optimization algorithms may be implemented to maximize or minimize an objective function. Thus, for example, DBS optimization algorithms may be implemented in an effort to find the best solution from feasible solutions.

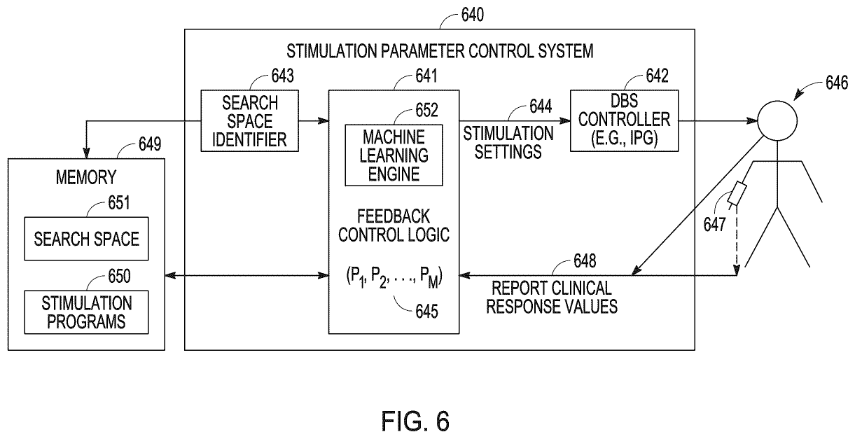

FIG. 6 illustrates, by way of example and not limitation, a stimulation parameter control system and a part of the environment in which it may operate. The stimulation parameter control system 640, which may be implemented as a part of the processor 427 in FIG. 4, may include a feedback control logic 641, a DBS controller 642, and a search space identifier 643. DBS is used as an example. It is noted that the system may be implemented for other stimulation therapies such as, but not limited to, SCS or PNS. The feedback control logic 641 may be implemented in, for example, the CP 104 or the RC 103 in FIG. 1. The feedback control logic 641 may determine or modify one or more stimulation settings 644 for a stimulation lead at a target stimulation region, such as a region in a brain hemisphere. A stimulation setting may include an electrode configuration and values for one or more stimulation parameters (P₁, P₂, . . . , Pₘ) 645. The electrode configuration includes information about electrodes (ring electrodes and/or segmented electrodes) selected to be active for delivering stimulation (ON) or inactive (OFF), polarity of the selected electrodes, electrode locations (also referred to as contact locations, which may include longitudinal positions of ring electrodes along the length of a lead, or angular positions of segmented electrodes about a circumference of a cross-section of the lead at a longitudinal position), and stimulation modes (e.g., monopolar pacing or bipolar pacing), etc. The stimulation parameters may include, for example, current amplitude values, current fractionalization across electrodes, stimulation frequency, stimulation pulse width, etc. In some examples, the feedback control logic 641 may modify the stimulation setting 644 such as by changing a stimulation parameter value or modifying an electrode configuration.

The stimulation setting 644 may be provided to the DBS controller 642 to configure the IPG or ETM to deliver DBS therapy to the patient 646 in accordance with the stimulation setting or the modified stimulation setting. The stimulation may produce certain therapeutic effects and/or side effects on the patient. Such therapeutic effectiveness and side effects, also referred to as clinical responses or clinical metrics, may be provided to the feedback control logic 641. In an example, the clinical responses may be based on patient or clinician observations. For example, motor symptoms such as bradykinesia (slowness of movement), rigidity, tremor, among other symptoms or side effects, may be scored by the patient or by the clinician upon overserving or questioning the patient. In some examples, the clinical responses may be objective in nature, such as measurements automatically or semi-automatically taken by a sensor 647. In an example, the sensor 647 may be included in a wearable device associated with patient 646, such as a smart watch. For example, a Parkinson's patient may be fitted with a wearable sensor that measures tremors, such as by measuring the frequency and amplitude of such tremors.

The clinical responses, either reported by the patient or measured by a sensor, may be converted to clinical response values 648, also referred to as clinical response scores. In an example, the clinical response values 648 may be computed based on the intensity, frequency, or duration of one or more of tremor, rigidity, or bradykinesia responses. Based upon the received clinical response values 648, the feedback control logic 641 may adjust electrode configurations or values of one or more stimulation parameters 645. The feedback control logic 641 may send the adjusted (new or revised) stimulation setting 644, such as the electrode configuration or the adjusted stimulation parameter values, to further configure the DBS controller 642 to change the stimulation parameters of the leads implanted in patient 646 to the adjusted values.

The feedback-control loop may continue until an optimal, desired, or acceptable outcome is reached, such as maximizing therapeutic effectiveness while minimizing unwanted side effects, or until a specific stop condition is reached such as number of iterations, time spent in programming session, or the like. An outcome may be considered optimal, desired, or acceptable if it meets certain threshold values or tests (e.g., improved clinical response for the patient, faster programming of the device, increased battery life, and/or control multiple independent current sources and directional lead). Such an iterative process of looking for a stimulation setting (e.g., an electrode configuration and stimulation parameter values for the electrode) is referred to as a stimulation setting optimization process. The outcome being reached may be referred to as an optimization criterion, and the resultant stimulation setting may be referred to as an optimal base stimulation setting (BSS). By way of example and not limitation, the optimization criterion may include possible optimal clinical outcome within the parameters chosen; time spent, iterations taken, or power usage to explore the search space until a desired clinical outcome is reached (assuming multiple outcomes with the same or comparable clinical response); among others.

In an example, the optimization criterion includes the clinical response values exceeding a threshold value or falling into a specified value range, indicating a satisfactory therapeutic outcome has reached. Depending on how the clinical response values are computed, one or more optimal base stimulation settings may be determined. For example, the clinical response values may be computed using a single response effect (e.g., one of bradykinesia, tremor, or rigidity). The optimal base stimulation settings (e.g., BSS₁ through BSS₄), may be stored in the memory 649. In an example, a stimulation setting, along with the corresponding unique clinical response indicator (e.g., weighted combination of clinical effects with unique weight factors) form a stimulation program 650, which may also be stored in the memory 649. Each stimulation program may be associated with, or tagged by, one or more unique clinical response indicators. In some examples, the clinical response values 648 may be weighted according to the time at which the test took place.

In various examples, the stimulation parameter control system 640 may be executed on its own and is not connected to a controller. In such instances it may be used to merely determine and suggest programming parameters, visualize a parameter space, test potential parameters, etc.

The process of searching for a stimulation setting (e.g., an electrode configuration and/or stimulation parameter values) typically involves significant computation and time, especially when electrode configuration involves segmented electrodes in a directional lead. If testing all possible settings in the entire parameter space (including electrode configurations and combinations of stimulation parameter values) is done as comprehensively as possible, stimulation would need to be provided to the patient for each possible setting, which may end up with a burdensome and time-consuming programming session. Because practically a programming session may only last a few hours, only a fraction of possible electrode configuration and stimulation parameter combinations may reasonably be tested and evaluated. To reduce the time taken and to improve the efficiency of stimulation setting optimization process, a reduced or restricted electrode configuration and parameter search space may be used. By applying limitations or constraints to the electrode configurations and parameter values, the restricted search space may include a subset of electrodes (e.g., a subset of ring electrodes and/or a subset of segmented electrodes on a lead) that are selected as active electrodes for delivering stimulation, and values or value ranges for one or more stimulation parameters (e.g., a range of current amplitude ranges for an active electrode). Stimulation setting optimization, when performed within such a search space, may be more efficient and cost-effective than searching through the entire parameter space for one or more optimal base stimulation settings.

The search space identifier 643 may automatically determine a search space 651 for a stimulation lead at a neural target, such as a region in a brain hemisphere, by imposing certain limitations or constraints on the electrode configurations and/or parameter values or value ranges. In an example, the search space 651 may be determined based on spatial information of the lead, such as lead positions with respect to neural targets, which may be obtained from imaging data of the lead and patient anatomy. Additionally, or alternatively, the search space 651 may be determined based on physiological information such as physiological signals sensed by the electrodes at their respective tissue contact locations. The physiological information may include patient clinical responses to stimulation. In some examples, prior knowledge about patient medical condition, health status, DBS treatment history may be used to determine the search space 651. In an example, the search space identifier 643 may exclude those electrodes on the lead that are out of a region of interest, such that the search space includes only those electrodes within the target of interest. One or more stimulation parameters may be restricted to take certain values or within value ranges. For example, the restricted search space may include certain electrode positions and value ranges for stimulation current amplitude, frequency, or pulse width. The feedback control logic 641 may determine one or more optimal base stimulation settings by searching through the identified search space 651. The identified search space 651 may be stored in the memory 649.

The feedback control logic 641 may include a machine learning engine 652 that may facilitate the stimulation parameter control system 640 (or a user of the system) to explore the search space in order to choose values for programming the DBS controller 642. The machine learning engine 652 may employ supervised or unsupervised learning algorithms to train a prediction model, and use the trained prediction model to predict patient clinical responses to an untested stimulation setting (e.g., untested stimulation parameter values or untested electrode configurations), or to estimate or predict stimulation parameters values or electrode configurations that, when provided to the DBS controller 642 to deliver stimulation accordingly to the patient 646, would produce desired or improved clinical responses. Examples of the learning algorithms include, for example, Naive Bayes classifiers, support vector machines (SVMs), ensemble classifiers, neural networks, Kalman filters, regression analyzers, etc. The machine learning engine 652 may build and train a prediction model using training data, such as stimulation parameter values and corresponding patient clinical responses. The training data may be acquired from a training session such as performed in a clinic. Additionally, or alternatively, the training data may be obtained from historical data acquired by the stimulation parameter control system 640. With its learning and prediction capability, the machine learning engine 652 may aid a user (e.g., a clinician) in exploring the stimulation parameter space more effectively and more efficiently to produce results that are optimal, desired, or acceptable.

In some examples, the machine learning engine 652 may use imaging data to inform the choice of the next set of values, which may be used when the algorithm finds itself in a region of parameter space for which the clinical responses are not substantially affected by the changes in the stimulation parameters, and the choice of next step is not apparent from the patient response alone. Imaging data that provides information about the location of the lead in the patient's brain along with priors informing the algorithm of which directions may be better choices for the next step could lead to faster convergence.

In some examples, the machine learning engine 652 may determine expected outcomes for parameter values that have not yet been tested based upon what the machine learning engine 652 has “learned” thus far and provide a recommendation for a next set of values to test. Here, testing refers to the iterative testing required to find an optimal stimulation setting for configuring the DBS controller 642. The recommendation for a next set of values to test is based upon which of the determined expected outcomes meet a set of designated (determined, selected, preselected, etc.) criteria (e.g., rules, heuristics, factors, and the like). For example, rules considered may include such factors as: the next set of values may not be one of the last 10 settings tested or may not be too close to previously tested setting. Accordingly, the feedback control logic 641 with its machine learning engine 652 is used to systematically explore the stimulation parameter space based upon what it has learned thus far and (optionally) different rules and/or heuristics that contribute to achieving optimal outcomes more efficiently.

The process for determining expected outcomes for parameter values that have not yet been tested may involve use of other data for machine learning. For example, data from other programming sessions for the same patient as well as from other patients may be used to train the machine learning engine 652. In some examples, no prior data may be used. In this case, the machine learning engine 652 may use data learned from this patient only in one particular setting. In other examples, data from the same patient but from previous sessions may be used. In some examples all patient data from all sessions may be used. In some examples all patient data utilizing lead location information (knowledge of lead location in space relative to anatomy) may be used. Different other combinations are also possible.

In order to use this data for machine learning purposes, the data may first be cleansed, optionally transformed, and then modeled. In some examples, new variables are derived, such as for use with directional leads, including central point of stimulation, maximum radius, spread of stimulation field, or the like. Data cleansing and transformation techniques such as missing data imputation and dimension reduction may be employed to prepare the data for modeling.

The machine learning engine 652 may determine how best a predicted outcome meets the optimal outcome metrics. Various optimization techniques may be used, examples of which may include but are not limited to: optimization algorithms and estimation procedures used to fit the model to the data (e.g., gradient descent, Kalman filter, Markov chain, Monte Carlo, and the like); optimization algorithms reformulated for search (e.g., simulated annealing); spatial interpolation (e.g., kriging, inverse distance weighting, natural neighbor, etc.); supplementary methods that aid the optimization process (e.g., variable selections, regularization, cross validation, etc.); other search algorithms (e.g., golden-section search, binary search, etc.). Using any of these techniques, the machine learning engine 652 may decide whether a particular predicted outcome for a set of stimulation parameter values is the fastest sufficing outcome, the best possible clinical outcome, or the optimal outcome with least battery usage, for example.

The feedback control logic 641 may be used to search and configure different types of stimulation parameters of the various leads potentially causing different clinical effects upon the patient. Examples of the stimulation parameters may include electrode configurations (electrode selection, polarities, monopolar or bipolar modes of stimulation), current fractionalization, current amplitude, pulse width, frequency, among others. Given these possible stimulation parameters, the stimulation parameter control system 640 may move about the parameter space in different orders, by different increments, and limited to specific ranges. In some examples, the stimulation parameter control system 640 may allow the user to provide search range limitations to one or more of the stimulation parameters to limit the range for that stimulation parameter over which the system will search for parameters. For example, the user may restrict which electrodes may be used for stimulation or may restrict the amplitude or pulse width to a certain range or with a selected maximum or minimum. As one illustration, based on the site of implantation, the user may be aware that the distal-most and proximal-most electrodes are unlikely to produce suitable stimulation and the user limits the range of electrodes to exclude these two electrodes.

For a lead with segmented electrodes, the number of possibilities for parameter selection may be very large when combinations of electrodes and different amplitudes on each electrode are possible. In some examples using a lead with segmented electrodes, the selection of electrodes used for stimulation may be limited to fully directional selections (i.e., selection of only a single segmented electrode) and fully concentric selections (i.e., all electrodes in a single set of segmented electrodes are active with the same amplitude). In other examples, the initial movement through parameter space may be limited to fully directional and fully concentric selections. After a set of stimulation parameters is identified using these limits, variation in the selection of electrodes may be opened up to other possibilities near the selection in the identified set of stimulation parameters to further optimize the stimulation parameters.

In some examples, the number of stimulation parameters that are varied and the range of those variations may be limited. For example, some stimulation parameters (e.g., electrode selection, amplitude, and pulse width) may have larger effects when varied than other stimulation parameters (e.g., pulse shape or pulse duration). The movement through stimulation parameter space may be limited to those stimulation parameters which exhibit larger effects. In some examples, as the stimulation parameter control system 531 proceeds through testing of sets of stimulation parameters, the system may observe which stimulation parameters provide larger effects when varied and focus on exploring variation in those stimulation parameters.

In some examples, the stimulation parameter control system 640 may include a user interface for visualizing exploration of the stimulation parameter space as the system determines new and better parameter values to test until a solution is determined that fits within certain designated thresholds or a stop condition is reached. In some examples of the stimulation parameter control system 640, the user interface is part of the feedback control logic. In other examples, the user interface may be part of another computing system that is part of the stimulation parameter control system 640 or may be remote and communicatively connected to the stimulation parameter control system 640. The user interface may present to a user (such as a clinician, physician, programmer, etc.) a visualization of the predicted expected outcomes for (some of) the stimulation parameter values not yet tested and a recommendation for the next set of stimulation parameter values to test.

In some examples where a deep brain stimulator is configured via the DBS controller 642 with at least one set of stimulation parameter values forwarded by the feedback control logic 641, the clinician may monitor the patient throughout the process and record clinical observables in addition to the patient 646 being able to report side effects. When a side effect is observed, the various search algorithms may take that fact into account when selecting/suggesting a next set of values to test. In some examples, for example, those that select contacts via monopolar review, other parameters may be changed until they cause a side effect, which case is noted as a boundary. For example, in monopolar review where amplitude is another stimulation parameter being varied, the amplitude may be increased progressively (e.g., via a variable ramp protocol) until a side-effect is observed.

In some examples, more than one clinical metric (e.g., tremor, rigidity, bradykinesia, etc.) may be important observables. Different examples of the stimulation parameter control system 640 may handle these metrics differently. For example, some examples might identify an ideal location for each metric and choose one ideal location between them, set in the patient's remote controller so the patient may choose as needed, or choose a best combined outcome. As another example, some examples may search multiple outcomes at the same time and use the best combined score as the best outcome or find a best location for each metric individually. As yet another example, some examples may use a sequential process for selecting stimulation parameter values for multiple outcomes. For example, a system may search parameter space for a first outcome (e.g., bradykinesia) and, upon finding a suitable end condition, then search parameter space for a second outcome (e.g., rigidity). While searching parameter space for the first outcome, clinical response values for both the first and second outcomes may be obtained. Thus, when the system switches to the second outcome there are already a number of clinical response values for that outcome which will likely reduce the length of the search.

In some examples, two stimulation leads may be implanted to produce stimulation effects on two sides of the body (e.g., the right and left sides of the body). The same procedure described herein may be used to either jointly determine the stimulation parameters for the two leads by exploring the joint parameter space or individually determine stimulation parameters for the two leads by exploring the parameter space for each lead individually. In some examples, the user may determine for each side of the body which clinical response is dominant or most appropriate. This may be done, for example, by having the patient perform a single task which captures multiple responses (e.g., connecting dots on the screen to monitor tremor and bradykinesia of the movement) or a small series of tasks. This enables the system to determine which clinical response to use to identify the stimulation parameters for that side of the body.

As noted, the feedback may be provided directly by the patient 646, entered by an observer such as a clinician (not shown), or may be provided by means of a sensor 647 associated with and in physical, auditory, or visual contact with the patient 646. Examples may include, but are not limited to, accelerometers, microphones, and cameras. In an example, the sensor 647 may be included in a wearable device associated with patient 646, such as a smart watch. In an example where the feedback may be monitored automatically or semi-automatically, such as with use of sensor 647, it may not be necessary for a clinician or other observer to be present to operate the stimulation parameter control system 640. Accordingly, in such examples a user interface may not be present in system 640.

In some examples, the stimulation parameter control system 640 may determine one or more optimal base stimulation settings using predicted clinical responses for untested stimulation parameter values or untested electrode configurations without actually delivering stimulation. Such base stimulation settings are referred to as estimated or predicted base stimulation settings, to distinguish from the tested base stimulation settings that are based on the tested clinical response (either reported by the patient or measured by a sensor) to actually delivered stimulation. For examples, based on the “tested” base stimulation settings, the stimulation parameter control system 640 may estimate an optimal base stimulation setting associated with a composite clinical response defined as x%* bradykinesia+y%*tremor+z%* rigidity, or simply denoted by the weight factors (x%, y%, z%). In an example, the stimulation programs 650 may be stored in a lookup table, where each tested or estimated base stimulation setting (e.g., BSS₁ through BSS₈) may be tagged by respective clinical response indicators or weight factors for clinical effects. In an example, the memory 649 may be a part of memory circuitry internal to the IPG. The RC or the CP may request access to the memory 649 to retrieve therefrom one or more stored stimulation programs 650 or the search space 651.

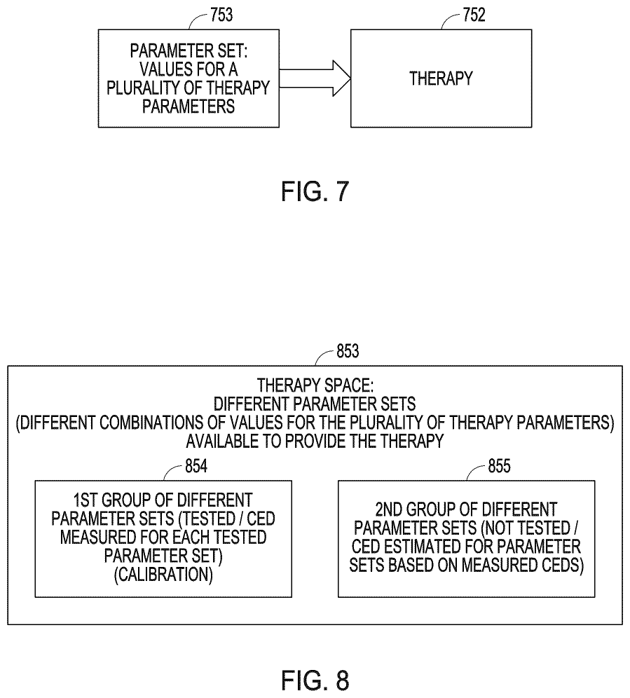

FIG. 7 illustrates a therapy 752 being delivered according to a parameter set. The parameter set may be programmed into the device to deliver the specific therapy using specific values for a plurality of therapy parameters. By way of example and not limitation, the therapy parameters that control the therapy may include pulse amplitude, pulse frequency, pulse width, and electrode configuration (e.g., selected electrodes, polarity and fractionalization). The parameter set includes specific values for the therapy parameters.

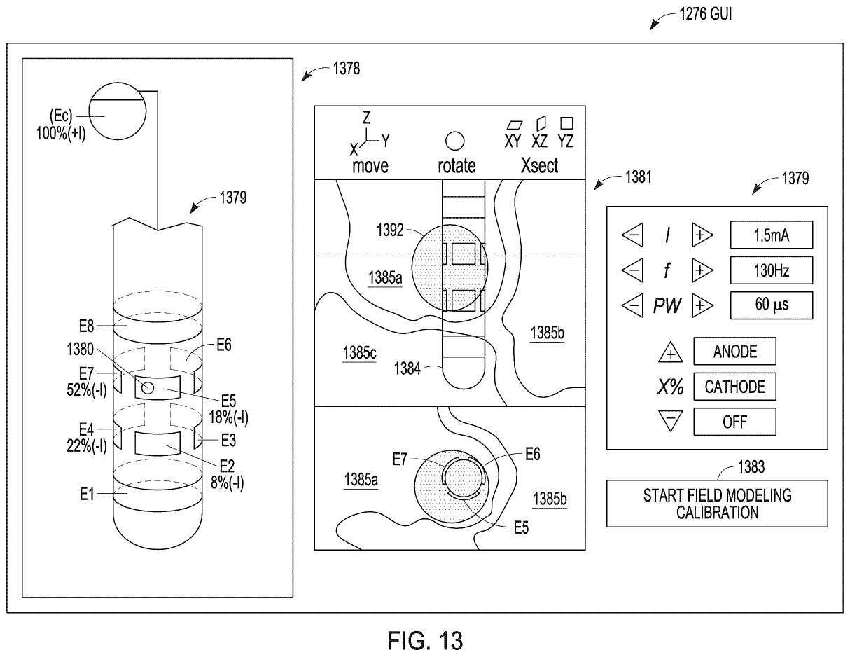

There may be different DBS optimization algorithms implemented to find a best solution from available, feasible conditions. A DBS optimization algorithm may be considered to be a process for improving the parameter sets until such time as the process ends. By way of example, the process may end if a resulting parameter set satisfies objective(s) of the therapy even though continuing with the optimization process may further improve the resulting parameter set. For example, some DBS optimization algorithms may test parameter sets within a search space and acquire clinical effect data and/or sensor data for each tested parameter set and creating a stimulation effects map (see, for example FIGS. 8-12), and some DBS optimization algorithms may attempt to overlap stimulation field models (SFMs) over a representation of targeted tissue (see, for example FIGS. 13-15). According to various embodiments, the regression model(s) may enhance the performance of these algorithms. For example, the regression model(s) may be configured to seed the search implemented by these algorithms or to determine when to stop the search process that is being implemented using these algorithms. Therefore, these DBS optimization algorithms search techniques are briefly discussed below to provide examples of DBS programming algorithms.

FIGS. 8-12 illustrate a DBS optimization algorithm by way of example and not limitation. FIG. 8 illustrates a therapy space, which includes different parameter sets potentially available for delivering the therapy. The different parameter sets 853 have unique combinations of values for the therapy parameters. The therapy space may be burdensomely large as there may be many unique combinations of values for therapy parameters (e.g., many unique parameter sets). Some parameter sets within the therapy space may be tested and the corresponding clinical effect data (CED) may be measured or otherwise acquired for the tested parameter sets. These tested parameter sets are illustrated as a first group of different parameter sets within the therapy space 854. Other parameter sets may not be tested (e.g., second group of different parameter sets 855). The CED for these parameter sets may be estimated based on measured CEDs for the patient or a patient population.

For example, CED may be directly measured to provide calibration settings. The therapy sessions may be delivered using different therapy settings, and the CED may be recorded for each session. For a neurostimulator such as DBS, SCS, PNS or TENS, for example, the therapy may involve delivering electrical waveforms, which may be a pulsed waveform. Programmable settings for the pulse waveform may include a pulse amplitude, a pulse width, a pulse frequency, a pulse train duration, a pulse-to-pulse duty cycle, a pulse train to pulse train duty cycle (stimulation ON/OFF), and a stimulation schedule (e.g., programmable start and/or stop times, such as but not necessarily a calendar-based schedule). The programmable settings may further include controlling which of a plurality of electrodes are active and which are off, the polarity of each active electrode (which active electrode(s) are anode(s) and which are cathode(s), and the contributions (e.g., electrode fractionalization) of total energy delivered to individual one(s) of the anode(s) and individual one(s) of the cathode(s). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. Controlling the individual contributions by individual electrodes adjusts the location and shape of the stimulation field, to modulate different combinations of neural elements. The settings may be spread throughout the stimulation space for use in identifying clinical responses from session to session, including both the previously-measured clinical effects for tested stimulation settings and predicted or estimated clinical effects for stimulation settings that were not previously tested.

Given the large number of available parameters sets, it is not practical to test all possible parameter sets to map the corresponding stimulation effects for the parameter sets. However, based on what has been tested, stimulation effect predictions (e.g., estimated responses) may be made for parameter sets that were not tested. By way of example and not limitation, these predictions may be used to determine a good parameter set for testing or for therapy.

The responses or stimulation effects for tested parameter sets may include patient responses associated with patient outcomes which may be referred to as clinical effects data. Patient outcomes may include perception, therapeutic effects, and side effects. The responses or stimulation effects for tested parameter sets may also include sensed data. For a system that is capable of sensing, not only are only some of the available stimulation sets tested, but sensing may have been performed for only some of the tested stimulation sets. The sensing data for these parameter sets may be used to extrapolate or interpolate sensed data predictions for parameter sets that are not associated with sensed data. Some of the tested stimulation sets may be associated with patient outcomes such as patient symptoms (e.g., clinical effects data). Some of the tested stimulation sets may be associated with both sensed data and patient outcomes, some tested stimulation sets may be associated with only patient outcomes without patient data, and some tested stimulation sets may be associated with only sensed data without patient outcomes. A stimulation effects map may include a combination of sensing data and patient outcomes, both tested and predicted, which may provide more information about stimulation parameter sets that should be tested or that may even be used in a program to treat the patient.

There may be many parameters in a set that may be tested. In order to assist with visualization, the different parameter sets may correspond to different stimulation locations, and some of these stimulation locations may be tested. These tested locations may be used to make predictions for other locations.

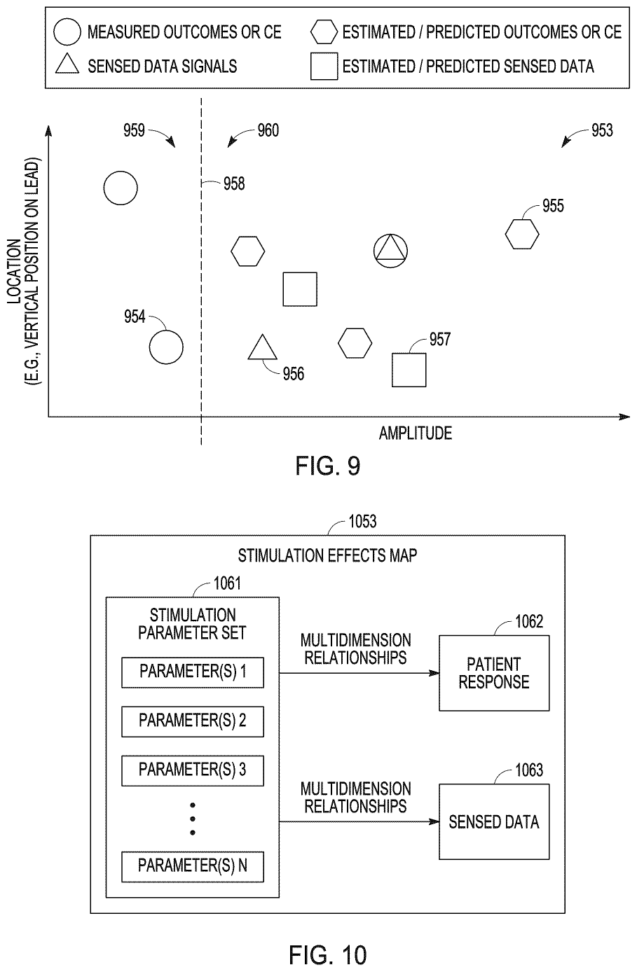

FIG. 9 illustrates, by way of example and not limitation, a two-dimensional plot of location and amplitude for measured and estimated outcomes (e.g., clinical effects) and sensed and estimated data. This map 953 combines both the amplitude of the neurostimulation on the X-axis and the position of the neurostimulation on the lead on the Y-axis. It is noted that the map may include information for additional stimulation parameter such as, but not limited to, pulse width and pulse rate. Therefore, multidimensional maps may be implemented.

In the illustrated map 953, the circles 954 represent measured, observed or experienced patient outcomes (e.g., clinical effects) for at least some tested parameter sets and the hexagons 955 represent estimated patient outcomes for at least some untested parameter sets. The patient outcomes may be entered in response to queries on a user device. The patient outcomes may be a side effect, a therapeutic effect, or other outcome. Thus, for example, the patient outcomes may be improvements in a symptom and/or a side effect. By way of example and not limitation, the circles may represent motor testing results for various DBS therapy parameter sets. The triangles 956 may represent the sensed data for some tested stimulation parameter sets. and the squares 957 represent estimated sensed data for at least some of the untested parameter sets.

The dotted line 958 may represent a threshold for the stimulation effect that is being mapped, where points on one side 959 represent parameter combinations that do not produce the stimulation effect on points on the other side 960 represent parameter combinations that do produce the stimulation effect. For a simpler two-dimensional map, the dotted line may simply be illustrated as a vertical line. However, the line may have different slopes and need not be a straight line but rather may have various inflections. Multi-dimensional maps may have a multidimensional function separating parameter sets that do not provide the stimulation effect from parameter sets that do provide the stimulation effect.

FIG. 10 illustrates, by way of example and not limitation, a stimulation effects map 1053 having multidimension relationships between a stimulation parameter set 1061 (illustrated as including N parameter(s)) and each of a patient response 1062 and sensed data 1063. Stimulation effects (e.g., a patient response and/or sensed data) may be associated with different stimulation parameter sets with three or more stimulation parameter(s). The parameter set includes “parameter(s)” that may be associated with the patient response and/or sensed data, as the association may be for one parameter or for a composite parameter such as “charge” where charge corresponds to a combination of pulse amplitude and pulse width.

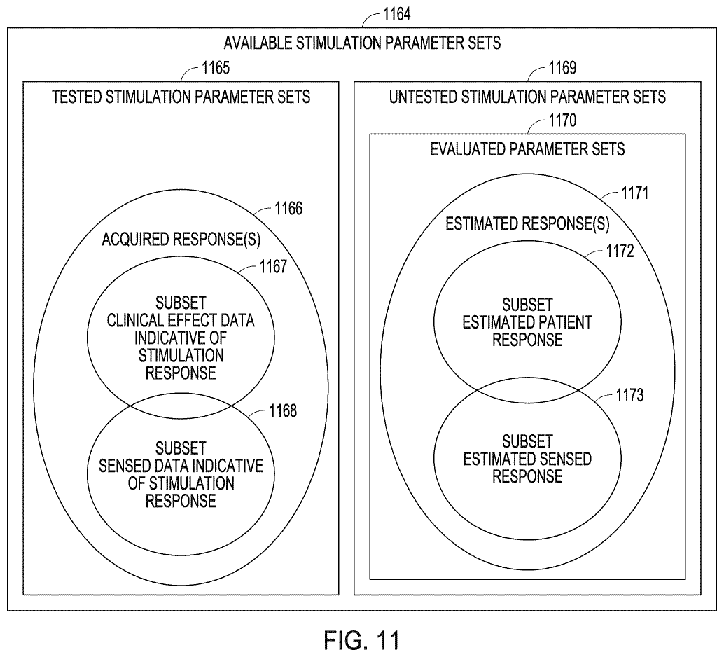

FIG. 11 illustrates, by way of example and not limitation, available stimulation parameter sets. The available parameter sets 1164 may include an electrode configuration and a stimulation waveform configuration. The electrode configuration may at least partially control the location of the stimulation field and may include parameters such as a selection of activated electrodes and the polarity of active electrodes fractionalization across the active electrode. The stimulation waveform configuration may include waveform parameters such as, but not limited to, amplitude, frequency and pulse width. Some of the available stimulation parameter sets 1164 are tested (see tested stimulation parameter sets 1165) by delivering neurostimulation using corresponding parameter values, and acquiring responses 1166 to the neurostimulation. The acquired responses 1166 for a tested parameter set 1165 may include a subset of clinical effect data 1167 indicative of a stimulation response (e.g., clinical effect) such as at least one patient symptom. The acquired responses 1166 for a tested parameter set 1165 may include a subset of sensed data indicative of a stimulation response 1168. By way of example and not limitation, sensed data may correspond to patient movement sensed using an accelerometer and/or camera. The acquired response subsets 1167 and 1168 may be exclusive or nonexclusive. For example, a single evaluated parameter set may only be associated with acquired clinical effect data 1167, only associated with acquired sensed data 1168, or may be associated with both acquired clinical effect data 1167 and acquired sensed data 1168. Some of the available stimulation parameter sets 1164 are not tested (see untested stimulation parameter sets 1169), and some of these untested stimulation parameter sets 1169 are evaluated 1170 by predicting or estimating the response(s) 1171. For example, the estimations may be based on extrapolating or interpolating the estimated response using acquired responses and may also use other estimated response(s). The estimated response(s) 1171 for an evaluated parameter set 1170 may include a subset of estimated patient response(s) 1172 and a subset of estimated sensed response(s) 1173. The estimated response subsets 1172 and 1173 may be exclusive or nonexclusive. For example, a single evaluated parameter set 1170 may only be associated with estimated patient response(s) 1172, only associated with estimated sensed response(s) 1173, or associated with both estimated patient response(s) 1172 and estimated sensed response(s) 1173. The number of acquired responses may be sufficient to interpolate or extrapolate to provide the estimated response(s).

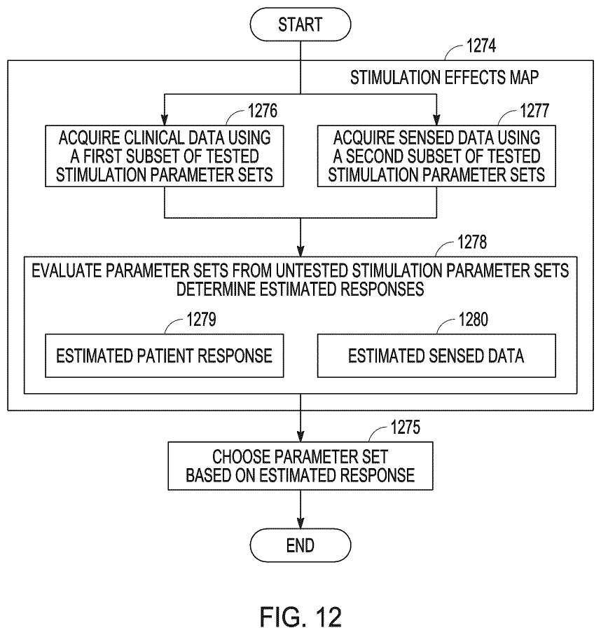

FIG. 12 illustrates, by way of example and not limitation, a method for providing a stimulation effects map 1274 which may be used to choose a parameter set 1275 to be tested or used for therapy. The map may be provided by testing stimulation parameter sets from a plurality of available stimulation parameter sets by controlling the neurostimulator for each of the tested stimulation parameter sets to deliver the electrical energy using the corresponding stimulation parameter set. The map 1274 may be provided by acquiring clinical effect data indicative of a patient response to the electrical energy delivered to the tissue using at least a first subset of the tested stimulation parameter sets 1276, acquiring sensed data indicative of a sensed response to the electrical energy delivered to the tissue using at least a second subset of the tested stimulation parameter sets 1277, and evaluating parameter sets from the plurality of untested stimulation parameter sets to provide evaluated parameter sets 1278. Each of the evaluated parameter sets 1278 may be provided by estimating at least one of the patient response 1279 or the sensed response 1280 to the electrical energy by interpolating, extrapolating or otherwise using the acquired clinical effect data 1276 and the acquired sensed data 1277. The stimulation effects map 1274 may include the acquired clinical effect data 1276, the acquired sensed data 1277, and the estimated responses for the evaluated parameter sets 1278.

In a nonlimiting example, the stimulation effects map 1274 may interpolate or extrapolate therapeutic thresholds or side effect threshold to track or predict therapeutic parameters or side effect parameters. By way of example and not limitation, the features of a sensed signal can be measured at a multitude of points (1277) and a predictive algorithm can be used to estimate these features at untested locations (e.g., 1280). The estimation may include interpolation and extrapolation via line and surface fitting. The estimation may be at least partially based on prior knowledge about expected topology of the stimulation response(s) within the search, parameter or other space, where the expected topology may include slopes and orientations, peaks, valleys, cliffs, or plateaus within the search space. In DBS, for example, a cliff in the therapy may be a certain amplitude of stimulation which causes a side effect and all amplitudes (or charge delivery) above the amplitude will also cause the side effect. A cliff may be identified using testing or determined anatomy using prior data from the current patient and/or a patient population.

Medical imaging may be used to determine anatomy. Anatomy may help inform that prior data for anything previously tested. For example, if a very low amplitude side effect is determined for one region, it may be predicted that lower amplitude side effects may occur at other regions of the lead as well. The lead may be orientated in a direction which could affect the dotted line in FIG. 10 or function demarcating the region where the stimulation effect occurs or where the region does not occur.

FIGS. 13-15 illustrate another DBS optimization algorithm by way of example and not limitation. FIG. 13 illustrates, by way of example and not limitation, a Graphical User Interface (GUI) operable on an external device where the GUI is capable of providing a clinician with a visual indication of how stimulation selected for a patient will interact with the tissue in which the electrodes are implanted. The GUI may be rendered on a clinician programmer, a patient external programmer or any other external device capable of communicating with the neurostimulator.

The GUI allows a clinician (or patient) to select the stimulation program that the neurostimulator will provide and provides options that control sensing of innate or evoked responses, as described below. The GUI 1376 may include a stimulation parameter interface 1377 where various aspects of the stimulation program can be selected or adjusted. For example, interface 1377 allows a user to select the amplitude (e.g., a current I) for stimulation; the frequency (f) of stimulation pulses; and the pulse width (PW) of the stimulation pulses. The stimulation parameter interface 1377 can be significantly more complicated to support more complex stimulation waveforms.

The stimulation parameter interface may allow a user to select the active electrodes—i.e., the electrodes that will receive the prescribed pulses. Selection of the active electrodes may occur in conjunction with a leads interface 1378, which can include an image 1379 of the one or more leads that have been implanted in the patient. The system may accommodate different lead designs.

The leads interface 1378 can include a cursor 1380 that the user can move (e.g., using a mouse connected to the clinician programmer) to select an illustrated electrode (e.g., E1-E8, or the case electrode Ec). Once an electrode has been selected, the stimulation parameter interface 1377 may be used to designate the selected electrode as an anode that will source current to the tissue, or as a cathode that will sink current from the tissue. Further, the stimulation parameter interface 1377 may allow the amount of the total anodic or cathodic current +I or −I that each selected electrode will receive to be specified in terms of a percentage, X. Two or more electrodes can be chosen to act as anodes or cathodes at a given time using independent current control, allowing the electric field in the tissue to be shaped. The currents specified at the selected electrodes can be those provided during a first pulse phase (if biphasic pulses are used), or during an only pulse phase (if monophasic pulses are used).

The GUI 1376 may include a visualization interface 1381 that allows a user to view an indication of the effects of stimulation, such as a stimulation field model (SFM) 1382 (which may also be referred to herein as a volume of tissue activated (VTA)) formed using the selected stimulation parameters. The SFM 1382 may be formed by field modeling. An example of creating an SFM based on stimulation parameters is discussed in U.S. Pat. No. 8,849,411, “USER-DEFINED GRAPHICAL SHAPES USED AS A VISUALIZATION AID FOR STIMULATOR PROGRAMMING”, assigned to Boston Scientific Neuromodulation Corporation, which is herein incorporated by reference in its entirety. Other examples of such modeling and volume estimation are discussed in U.S. Pat. No. 8,190,250 B2, entitled “SYSTEM AND METHOD FOR ESTIMATING VOLUME OF ACTIVATION IN TISSUE”, U.S. Pat. No. 8,706,250 B2, entitled “NEUROSTIMULATION SYSTEM FOR IMPLEMENTING MODELBASED ESTIMATE OF NEUROSTIMULATION EFFECTS”, U.S. Pat. No. 8,934,979 B2, entitled “NEUROSTIMULATION SYSTEM FOR SELECTIVELY ESTIMATING VOLUME OF ACTIVATION AND PROVIDING THERAPY”, U.S. Pat. No. 9,792,412 B2, entitled “SYSTEMS AND METHODS FOR VOA MODEL GENERATION AND USE”, all assigned to Boston Scientific Neuromodulation Corporation, which are incorporated by reference herein in their entirety. The illustrated embodiment of the GUI includes a selection option 1383 for initiating such modeling. Only one lead is shown in the visualization interface 1381 for simplicity, although again a given patient might be implanted with more than one lead. The visualization interface 1381 may provide an image 1384 of the lead(s) which may be three-dimensional.

The visualization interface 1381 may include tissue imaging information 1385 taken from the patient, represented as three different tissue structures 1385a, 1385b and 1385c that may comprise different areas of the brain for example. Such tissue imaging information may comprise a Magnetic Resonance Image (MRI), a Computed Tomography (CT) image or other type of image. Often, one or more images, such as an MRI, CT, and/or a brain atlas are scaled and combined in a single image model. The location of the lead(s) may be precisely referenced to the tissue structures because the lead(s) are implanted using a stereotactic frame (not shown). This allows the clinician programmer on which GUI is rendered to overlay the lead image 1384 and the SFM 1382 with the tissue imaging information in the visualization interface 1381 so that the position of the SFM 1382 relative to the various tissue present to the clinician's office to determine (or further refine) optimal stimulation parameters during a programming session.

Gauging the effectiveness of a given set of stimulation parameters typically involves programming the IPG with that set, and then reviewing the therapeutic effectiveness and side effects that result. Therapeutic effectiveness and side effects are often assessed by one or more different scores(S) for one or more different clinical responses, which are entered into the GUI of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst). Scores may also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI, and if necessary, converted to a score.

Quantitative objective information relating the position of the electrode lead with respect to anatomical features of the patient's brain may be used to predict stimulation parameters that should be effective for the patient and/or that are likely to cause side effects for the patient. The electrode leads may be implanted in, or near, a target brain structure, such as the patient's STN. Other brain structure targets may include the Globus Pallidus Internus, the Ventral Intermediate Nucleus, or other targets.

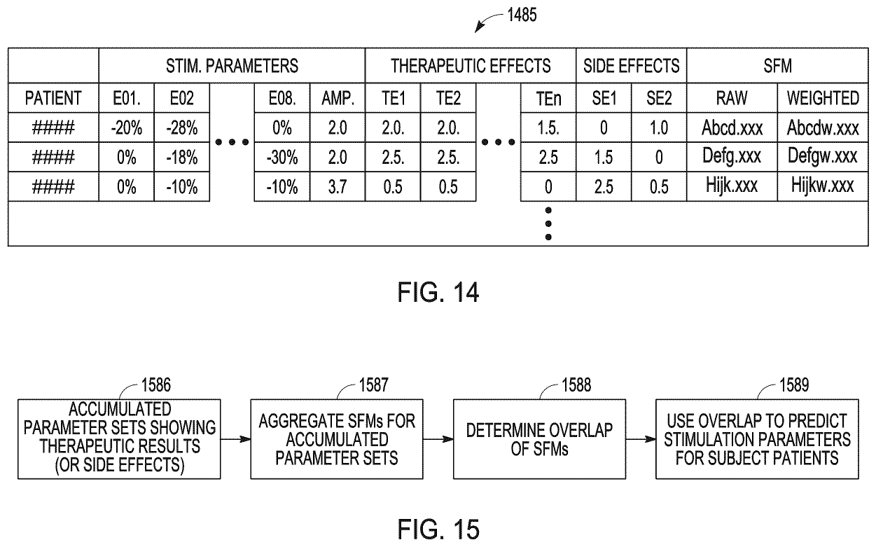

FIG. 14 illustrates, by way of example and not limitation, an accumulated database 1485 with data collected from fitting sessions conducted for a plurality of patients (and, possibly, for multiple hemispheres of the patients). The fitting data from the various fitting sessions includes data relating to the stimulation parameters that were applied to the patient. The stimulation parameters may include amplitude, stimulation field energy, frequency, pulse width, duty cycle, electrode configuration, or the like, may be collated in the database. In the illustrated example, amplitude (Amp.) and electrode configuration (i.e., the percentage of current delivered at each of the electrodes E01-E08) are shown. The stimulation according to each of the trial parameter sets may be scored based on its effect for the patient on the basis of patient responses to the stimulation, as is known in the art. According to some embodiments, the patient responses may include one or more of speech, tremor, rigidity, finger tapping, toe tapping, bradykinesia, hypokinesia, agility posture, gate, postural stability, or the like. In the illustrated example, such effects are referred to as “therapeutic effects,” (TE1-TEn). The accumulated database may also include scores for side effects for each of the trial parameter sets for the patient (shown as SE1 and SE2 in the illustration). Examples of side effects may include paresthesia, tremor, discomfort, and the like. The illustrated embodiment of the accumulated database also includes data for SFMs determined for each of the trial parameter sets used in the various fitting sessions for the various patients. In the illustration, the illustrated accumulated database comprises raw voxelized SFM data and weighted voxelized SFM data to emphasize or deemphasize voxels. The accumulated database provides correlations between trial parameter sets that provide good therapeutic outcomes (and/or that cause side effects) and SFMs for those trial parameter sets. Methods of associating SFMs with their therapeutic effects are also discussed in U.S. Pat. No. 11,577,069, the entire contents of which are incorporated herein by reference.

FIG. 15 illustrates, by way of example and not limitation, an algorithm 900 that can be used to predict effective therapeutic stimulation parameters (or stimulation parameters that may cause side effects) for a subject patient based on an accumulated database, as shown in FIG. 14. At 1586, the algorithm may determine stimulation parameter sets that have provided good therapeutic results (or side effects) during the fitting sessions of historic patients contained within the database. For example, the database may be scanned for parameter sets that were shown to be effective at treating symptoms similar to those presented in the subject patient. At 1587, SFMs of the identified parameter sets may be aggregated. The aggregation process may involve overlaying the voxelized SFMs, typically within a three-dimensional voxel space. At 1588, it may be determined where the aggregated SFMs overlap, i.e., determining where some number of the aggregated SFMs occupy the same volume within the voxel space. According to some embodiments, the determination of overlap may be based on a threshold. For example, the overlap region may be considered as a volume occupied by some threshold percentage of the aggregated SFMs. According to some embodiments, overlap determination may be determined using an automated aggregation algorithm. Visualizations (and/or mathematical analysis, for example, performed using an aggregation algorithm) of the overlap of aggregated SFMs for stimulation parameters associated with therapeutic results and/or side effects derived from the accumulated database can aid the programming of stimulation parameters for a subject patient. For example, the clinician may choose to try stimulation parameters for the subject patient that provide stimulation within a region where a high degree of overlap occurs in the aggregated therapeutic SFMs from the accumulated database (i.e., within the dark areas shown in FIG. 10A). In other words, the high overlap region may be considered a target volume for stimulation for the subject patient. Likewise, the clinician may seek to avoid stimulation parameters that stimulate within the high-overlap areas associated with side effects. That region may be considered an avoidance volume for the subject patient. The accumulated and overlayed SFMs may be projected upon representations of the subject patient's anatomy using a GUI. Such representations may be derived from imaging data, as is known in the art. Such overlays allow a clinician to correlate the areas of high overlap with specific portions of the patient's anatomy, for example, portions of the patient's STN that might be associated with effective therapy and/or with side effects.

The examples of DBS optimization algorithms described above with respect to FIGS. 8-12 and FIGS. 13-15 are nonlimiting examples of search algorithms that may be used to determine the parameter set that is to be used to deliver the therapy. The number of potential different parameter sets that may be processed by the search algorithm may be very large which may require a significant amount of time and processing resources. It is desirable to shorten the time to find the parameter set to be used to deliver the therapy. Benefits include less programming time for the patient and clinician, quicker implementation of the therapy corresponding to a desirable (e.g., “optimized”) parameter set, and less energy and processing resources for finding the desirable (e.g., “optimized”) parameter set.

The prediction of the potential clinical beneficial effect of the therapy and algorithm-based optimization of DBS settings may benefit from the aggregation of data from several patients e.g., generation of target and avoidance objects for image-guided programming. However, the aggregation of data such as the aggregation of SFMs may include steps like the normalization of anatomical spaces, that may add some uncertainty to the results. Additionally, current DBS optimization algorithms may only attempt to optimize a few parameters such as stimulation location and amplitude without exploring parameters such as pulse width and frequency.

Various embodiments of the present subject matter may use databases with information about the location of DBS leads and electrodes in relation to stimulation targets and the clinical effects of previously tested stimulation settings from several patients to train machine learning to generate regression models that aim predict the clinical effect of stimulation settings and lead locations.

Regression analysis is useful for understanding the relationships between variables to provide data-driven predictions. A regression model may be fit to the data to predict outcomes based on input variables. There are many types of regression analysis that may be used to mathematically describe relationships between variables. Further, it is noted that there are many types of regression models including different types of linear models and different types of nonlinear models. Nonlimiting examples of types of regression models that may be implemented using machine learning include linear regression, polynomial regression, logistic regression, lasso regression, and ridge regression.

Various embodiments employ trained machine-learning model(s) to improve the prediction of clinical effects for untested stimulation parameter sets based on lead placement information and clinical history for the patient including both first clinical effect data corresponding to times when the patient is not being treated with a therapy and second clinical effect data corresponding to times when the patient is being treated with the therapy. The trained machine-learning model(s) may be employed with other search algorithms to improve the performance of those search algorithms. In some embodiments, the trained machine-learning model(s) is not employed with other search algorithms, but rather are able to provide the desirable set based on lead placement information and clinical history for the patient including both first clinical effect data corresponding to times when the patient is not being treated with a therapy and second clinical effect data corresponding to times when the patient is being treated with the therapy. The lead placement information may include information regarding a lead entry point. The lead placement information may include information regarding a lead trajectory. The lead placement information may include information regarding a lead orientation. The lead placement information may include information regarding a neural target. For example, lead placement information may be derived from the target. The lead placement information may include information regarding relative lead position with respect to neuroanatomy or anatomical landmarks. The lead placement information may include information regarding anatomical coordinates defined by an anatomy of the patient. The lead placement information may include information regarding an identifier of a medical facility or a medical provided that implanted the DBS lead(s). For example, the lead placement information may be inferred since certain medical facilities and/or medical provider consistently follow a surgical procedure such that the result of the procedure is a particular lead placement.

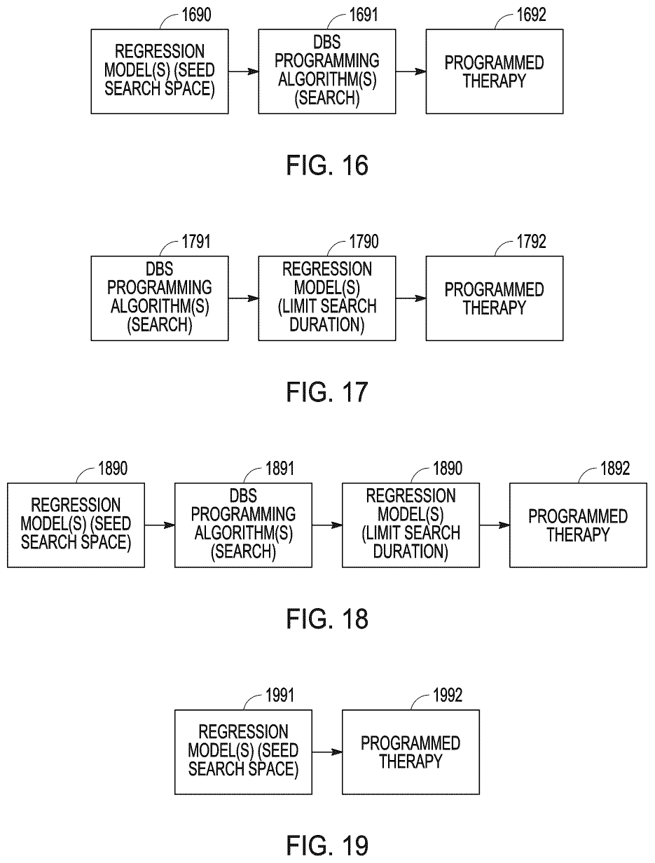

FIG. 16 illustrates, by way of example and not limitation, a method that uses trained machine-learning model(s) (e.g., regression model) 1690 to seed a search space for a DBS optimization algorithm 1691 to find the desired parameter set used in a programmed therapy 1692. By seeding the search space, the DBS optimization algorithm may begin processing parameter sets that are more similar to the desired or optimized parameter set that results from the DBS optimization algorithm, which allows the DBS optimization algorithm to determine the desired or optimized parameter set more quickly. Further, seeding the search space may allow the DBS optimization algorithm to consider stimulation parameters in addition to amplitude. For example, the pulse width and frequency may be optimized by the search process. The search process may be designed to optimize the active electrodes and/or the electrode fractionalization distributed among the active electrodes. Different stimulation waveform patterns (e.g., patterns with different pulse-to pulse intervals, different pulse amplitudes, different pulse widths, and/or pulse frequencies

FIG. 17 illustrates, by way of example and not limitation, a method that uses trained machine-learning model(s) (e.g., regression model) 1790 to determine when to end the search that is being performed by a DBS optimization algorithm 1791 to find the desired parameter set used in a programmed therapy 1792. The trained machine-learning model(s) may be used to more quickly reach the conclusion that further searching will not result in a significantly better parameter set than the current parameter set that is currently considered to be the optimized parameter set. Thus, the optimization process may be ended more quickly. Benefits may include less programming time for the patient and clinician, quicker implementation of the therapy corresponding to a desirable (e.g., “optimized”) parameter set, and less energy and processing resources for finding the desirable (e.g., “optimized”) parameter set.

FIG. 18 illustrates, by way of example and not limitation, a method that uses trained machine-learning model(s) (e.g., regression model) 1890 to seed a search space for a DBS optimization algorithm 1891 to find the desired parameter set used in a programmed therapy and that uses trained machine-learning model(s) (e.g., regression model) 1890 to determine when to end the search that is being performed by a DBS optimization algorithm 1891 to find the desired parameter set used in a programmed therapy 1892. Thus, the machine-learning models allows the DBS optimization algorithm to begin the search process with parameter sets that are more similar to the desired or optimized parameter set that results from the DBS optimization algorithm, and to more quickly reach the conclusion that further searching will not result in a significantly better parameter set.

FIG. 19 illustrates, by way of example and not limitation, a method that uses trained machine-learning model(s) (e.g., regression model) to directly find the desired parameter set used in a programmed therapy. Thus, as the model(s) 1991 themselves are trained with data, it is expected that the model(s) may be used to determine the desired parameter set to provide the therapy 1992 without implementing DBS optimization algorithms to search for the desired parameter set.

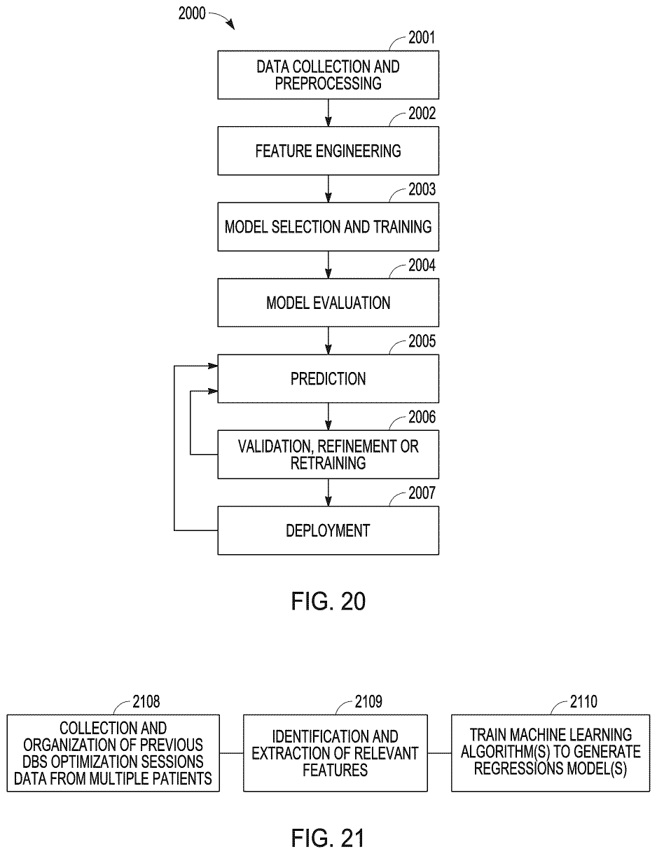

FIG. 20 is a flowchart depicting machine-learning pipeline 2000, according to some examples. The machine-learning pipeline 2000 may be used to generate a trained model. Broadly, machine learning may involve using computer algorithms to automatically learn patterns and relationships in data, potentially without the need for explicit programming. Machine learning algorithms can be divided into three main categories:
supervised learning, unsupervised learning, and reinforcement learning. Supervised learning involves training a model using labeled data to predict an output for new, unseen inputs. Examples of supervised learning algorithms include linear regression, decision trees, and neural networks. Unsupervised learning involves training a model on unlabeled data to find hidden patterns and relationships in the data. Examples of unsupervised learning algorithms include clustering, principal component analysis, and generative models like autoencoders. Reinforcement learning involves training a model to make decisions in a dynamic environment by receiving feedback in the form of rewards or penalties. Examples of specific machine learning algorithms that may be deployed, according to some examples, include various types of regression algorithms, which are a type of supervised learning algorithm. The performance of machine learning models is typically evaluated on a separate test set of data that was not used during training to ensure that the model can generalize to new, unseen data. Regression algorithms aim at quantifying some items (for example, by providing a value that is a real number). Generating a trained machine-learning program may include multiple phases that form part of the machine-learning pipeline 2000. A data collection and preprocessing 2001 phase may include acquiring and cleaning data to ensure that it is suitable for use in the machine learning model. This phase may also include removing duplicates, handling missing values, and converting data into a suitable format. A feature engineering 2002 phase may include selecting and transforming the training data to create features that are useful for predicting the target variable. Feature engineering may include receiving features (e.g., as structured or labeled data in supervised learning) and/or identifying features (e.g., unstructured, or unlabeled data for unsupervised learning) in training data. A model selection and training 2003 phase may include selecting an appropriate machine learning algorithm and training it on the preprocessed data. This phase may further involve splitting the data into training and testing sets, using cross-validation to evaluate the model, and tuning hyperparameters to improve performance. A model evaluation 2004 may include evaluating the performance of a trained model on a separate testing dataset. This phase can help determine if the model is overfitting or underfitting and determine whether the model is suitable for deployment. A prediction 2005 involves using a trained model to generate predictions on new, unseen data. A validation, refinement or retraining 2006 phase may include updating a model based on feedback generated from the prediction phase, such as new data or user feedback. A deployment 2007 phase may include integrating the trained model into an application or a more extensive system.

FIG. 21 illustrates, by way of example and not limitation, a process for creating machine-learning models that may be used to predict parameter sets from untested stimulation settings. At 2108, previous DBS optimization sessions from multiple patients may be collected and organized, similar to data collection and preprocessing 2001 in FIG. 20. At 2109, relative features from the data may be identified and extracted, similar to feature engineering 2002 in FIG. 20. At 2110, these features from the collected data may be used to train the machine learning algorithm(s) to generate regression models, which may be similar to one or more of model selection and training 2003, model evaluation 2004, and validation, refinement or retraining 2005.

The database used to generate the regression models may include information about the patient clinical state without therapy. In an example of using DBS as a therapy for Parkinson's Disease, the information may include Unified Parkinson's Disease Rating Scale III (UPDRS-III) Scores without therapy (e.g., patient in Off medication state). The database used to generate the regression models may include information about the patient clinical state with therapy other than DBS (e.g., UPDRS-III Scores with therapy (e.g., patient in On medication state). The database used to generate the regression models may include information about features of lead location in relation to stimulation target. By way of example, the lead location features may include lead angles in relation to principal axes of a Subthalamic Nucleus (STN). The database used to generate the regression models may include information about previously tested stimulation settings. For example, this information may include the location and amplitude of stimulation and their corresponding clinical effect (e.g., UPDRS-III based scores). The database may include the angle, orientation, and insertion site for the lead and the fractionalization of the electrodes that control the shape, size and direction of the stimulation field. The database may include other stimulation parameters such as, but not limited to, one or more of amplitude, pulse width and frequency.

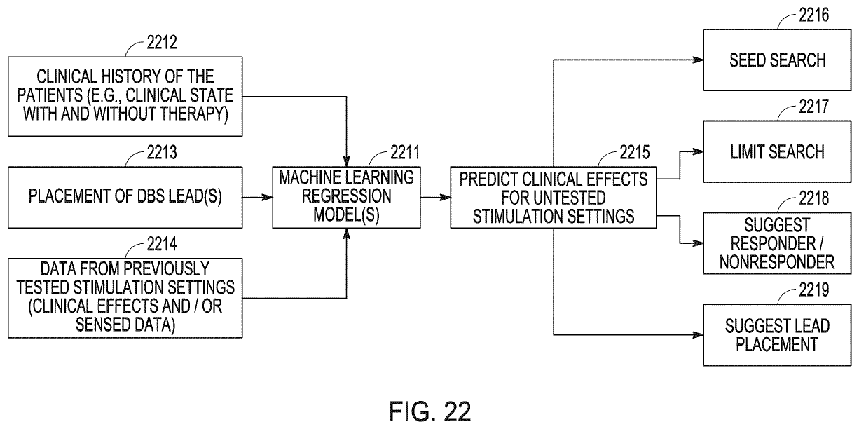

FIG. 22 illustrates, by way of example and not limitation, a process for using trained machine learning regression model(s) to predict clinical effects for untested stimulation settings. The model(s) 2211 may receive information about the clinical history of patient(s) 2212, including the clinical state of the patient with and without a therapy. The therapy may be a medicinal therapy, a DBS therapy, or other therapy. The model(s) 2211 may receive information about the placement of the DBS leads 2213. This information may include the location in relation to stimulation target and may include lead angles in relation to principal axes of a targeted tissue or other anatomical landmark, the orientation and insertion location of the lead, and the fractionalization of active electrodes on the lead, which control the location, size and shape of the stimulation field with respect to the lead. The model(s) may receive information about previously tested stimulation settings 2214, which may include the clinical effects and/or sensed data associated with a corresponding tested stimulation setting. The machine learning regressions models 2211 may be trained to predict the clinical effects of delivering therapy 2215 using untested stimulation settings. These predicted stimulation effects 2215 may be analyzed and used to seed a search for an optimization algorithm 2216 and/or to limit a search by an optimization algorithm 2217. These predicted stimulation effects 2215 may be analyzed and used to suggest whether a patient is a responder and will likely experience a desirable result from the therapy or is a non-responder 2218 and will likely not experience the desirable result from the therapy. Such information may be used to determine whether to continue with the DBS therapy. These predicted stimulation effects may be analyzed and used to suggest lead placement 2219 for the DBS therapy (e.g., lead insertion site, angle, orientation, and fractionalization.

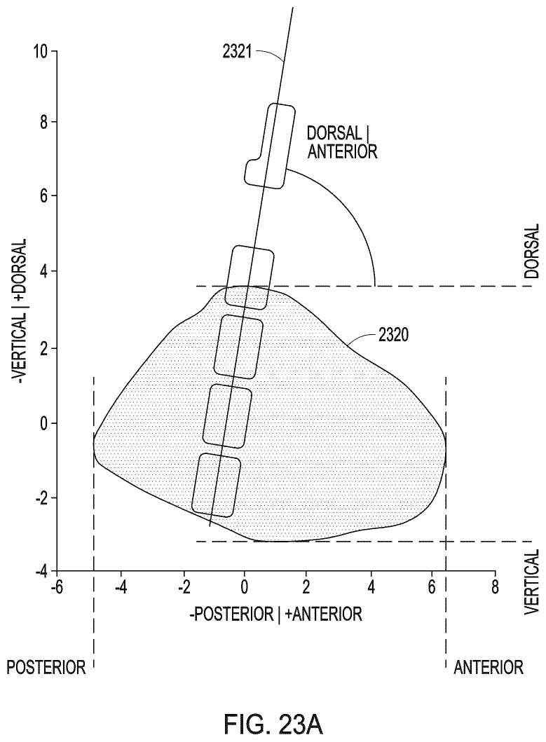

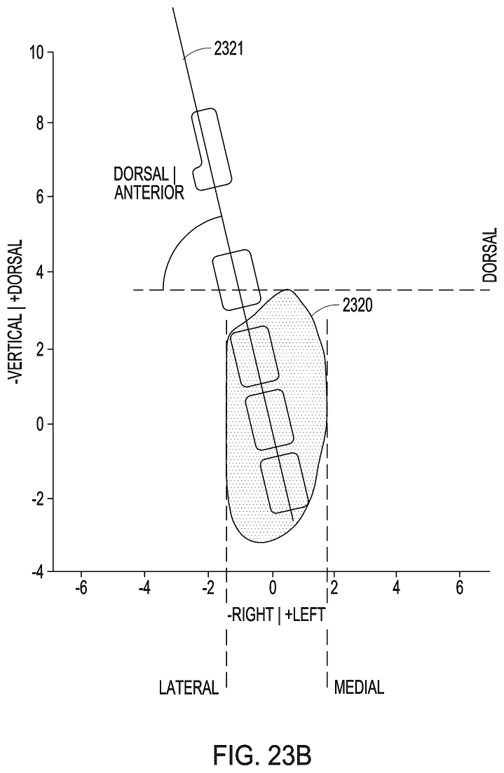

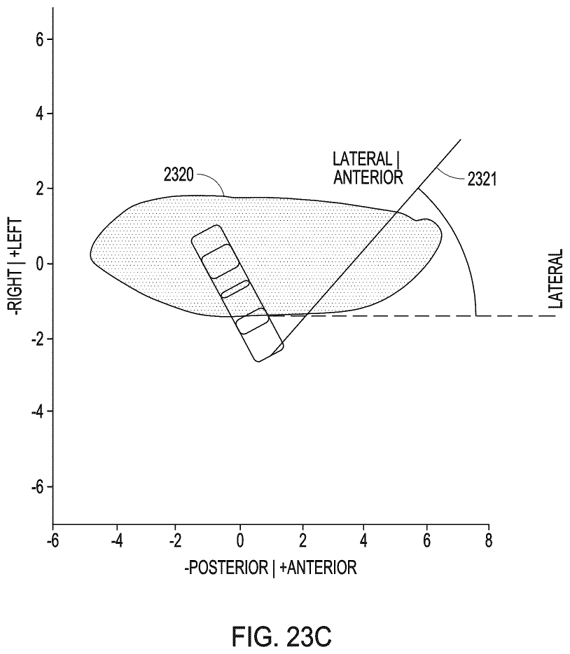

FIGS. 23A-23C illustrate, by way of example and not limitation, imaging of a lead with respect to targeted tissue such as the STN. FIG. 23A illustrates a mostly lateral view of the STN, and further represents a dorsal-anterior angle of the lead with respect to a dorsal axis. FIG. 23B illustrates mostly a frontal view of the STN, and further represents the dorsal/lateral angle of the lead with respect to a dorsal axis. FIG. 23C illustrates mostly a top view of the STN, and further illustrates a lateral/anterior angle with respect to a lateral axis. Each of FIGS. 23A-23C illustrate an STN image, a lead image, and reference angle(s) between the lead and at least one of the principal axes. The STN 2320 is a nonlimiting example, as other tissue may be targeted. The figures also illustrate a lead 2321 with electrodes thereon. The STN has a tilted orientation compared to the principal axes of the human body, and three principal axes may be used to describe a length, height and width of the STN. In an effort to normalize data for different patients, the lead angle may be defined based on the stimulation target (e.g., STN) using three principal axes that are used to describe a length, height and width of the stimulation target. There may be other ways to define the lead to at least one other reference. For example, the STN is not easily identified using MRI scans, but there may be other structures that are more easily identified such as, but not limited to, the red nucleus that is a structure in the ventral midbrain and is fairly close to the STN. External reference point(s) may be used to define the lead angle. For example, a stereotactic DBS surgical procedure uses a frame to stabilize the head and provide coordinates to help guide the lead into position in the brain. The positions for both the stimulation target (e.g., STN position and orientation) and the lead may be referenced to the frame.

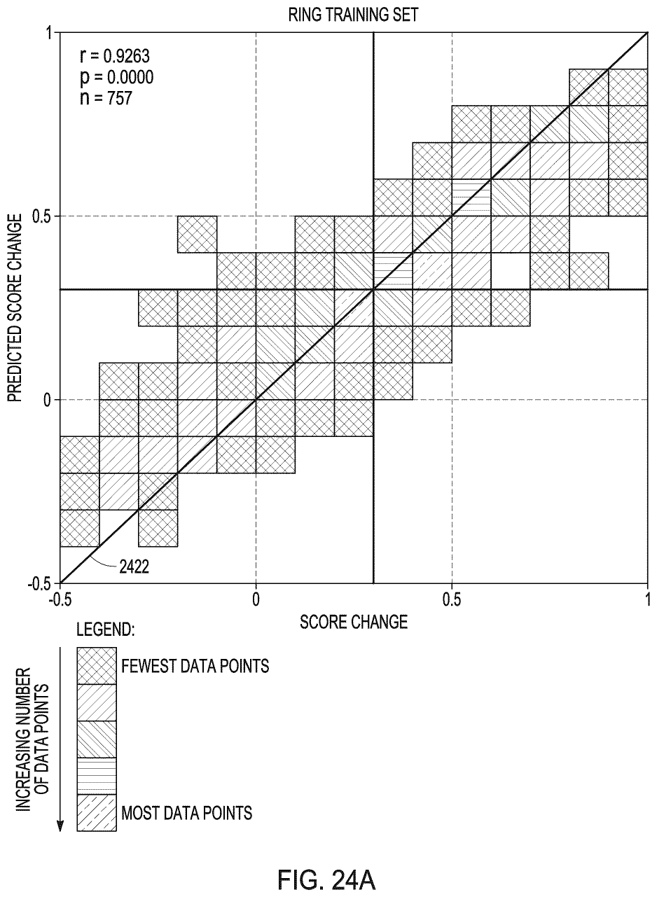

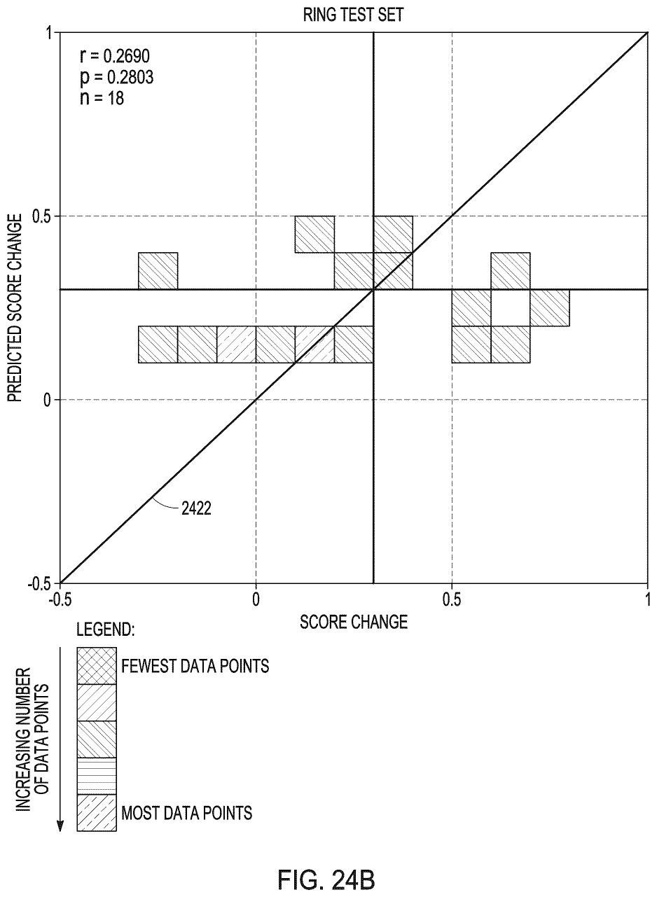

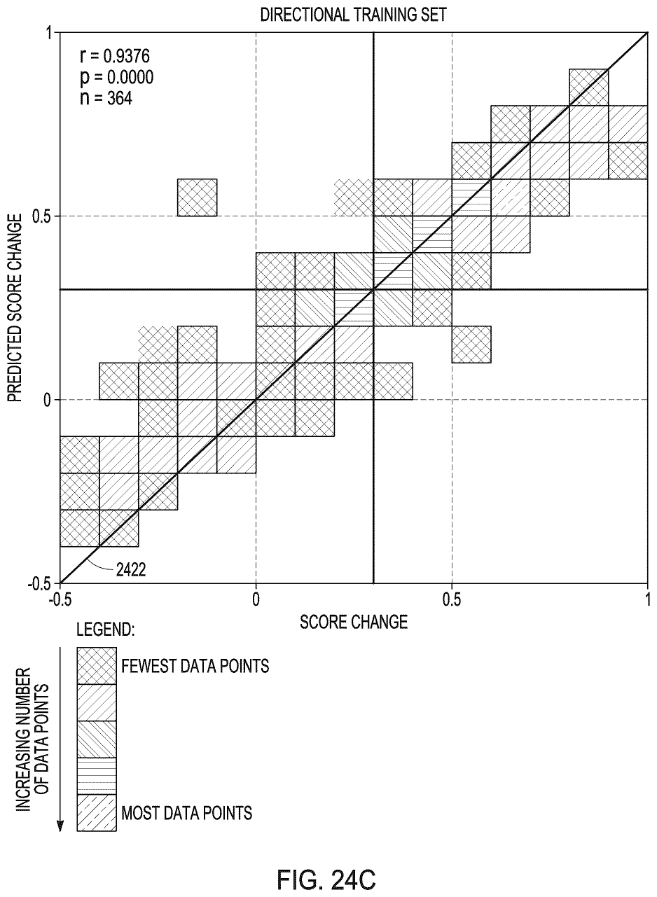

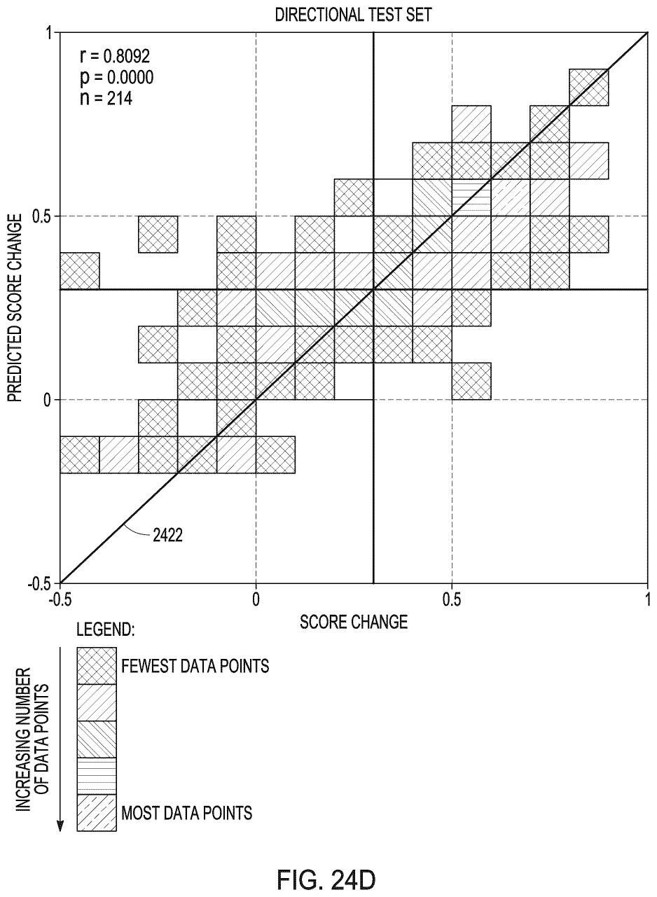

FIGS. 24A-24D illustrate, by way of example and not limitation, training data sets used to train a model and test data sets for evaluating the model that provide a proof of concept for training a regression model using data from many patients to train the model to predict clinical effects based on lead location and clinical effect data with and without therapy, and using other data from patients to test the model. The training and test sets for a ring electrode arrangement are shown in FIGS. 24A-24B, respectively. The ring electrode arrangement provides equal stimulation energy for all of the segmented electrodes in one row of the lead. The training and test sets for a directional lead arrangement are shown in FIGS. 24C-24D, respectively. The directional lead arrangement does not provide equal stimulation to all of the segmented electrodes in one row of the lead, which results in a directional field. Rather, by way of example, one of the segmented electrodes may have all or the largest fractional energy compared to the other segmented electrodes.

The vertical axis represents a predicted score change for a clinical effect and the horizontal axis represents the actual score change for the clinical effect. The diagonal line 2422 with a slope of 1 represents where the predicted score change perfectly corresponds to the actual score change. The darker color grids represent fewer data points and the lighter color grids represent more data points. It can be seen that, in both the ring and directional training sets, most of the data points cluster near the diagonal line indicating that the actual score changes closely followed the predicted score change. Similarly, in both the ring and directional test sets, most of the data points cluster near the diagonal line 2422. Thus, the predicted score changes closely follow the actual score changes. The variable r represents a measurement of correlation between variables where an r of 1 indicates that the variables are the same. The variables r, p and n are provided for each of the grids. The variable p provides a measure of the likelihood that the results are by chance where a low p indicates that the results are unlikely the result of chance. The number n represents the number of data points.

Similar charts may be implemented with threshold lines for the predicted/actual clinical effects. In the illustrated changes, the thresholds are set at 0.3. Other values may be used. Datapoints above the threshold may indicate a desirable result to the stimulation and datapoints below the threshold may indicate that a patient is a non-responder because the patient will not respond to the therapy. Thus, the regression model(s) may be used to predict whether the patient is a candidate for surgery or is not a candidate for surgery. The regression model(s) may be used to predict that a patient has a potential to get this score improvement. The regression model(s) may be used to provide the surgeon with more insight that the patient has the greatest potential to get a score improvement of at least a threshold using a particular lead orientation/location. The regression model(s) may be used to simulate results of stimulation using specific lead locations and/or orientations.

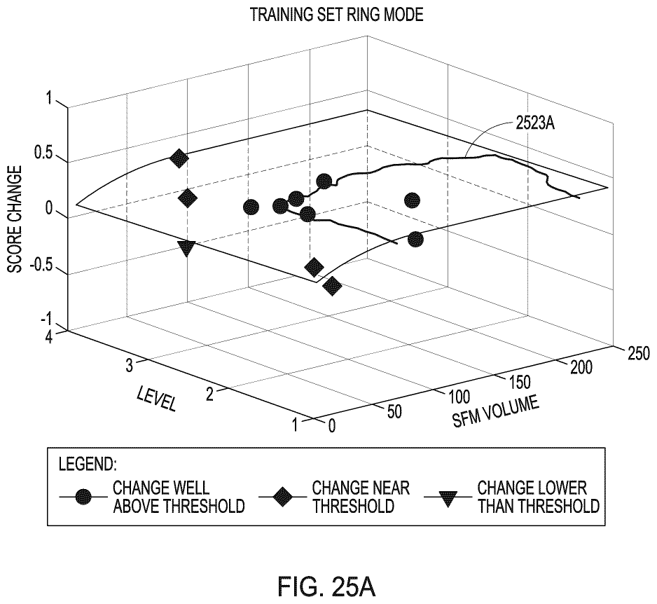

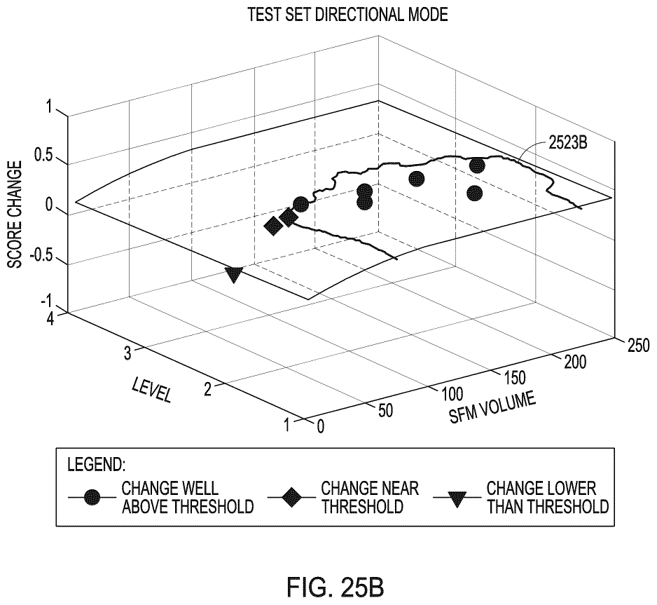

FIGS. 25A-25B illustrate, by way of example and not limitation, a training set for a ring mode and a test set for a direction mode, respectively. Each of these figures illustrate a three-dimensional plot includes a level axis which represents the level or row of electrodes on a lead, an SFM volume axis where larger number correspond to larger SFMs and also corresponds to larger amplitudes, and a score change axis. The threshold lines 2523A and 2523B on the surfaces represent thresholds where there is a high likelihood to get good results. Thus, the regression model(s) may be used to seed a DBS optimization algorithm. The threshold line may also be used to determine when to end a search (e.g., when most of the region within the threshold line has been considered). The figures include data points, including poor data points that result in an actual or predicted score change below a threshold, good data points that result in an actual or predicted score change near the threshold, or very good data points that result in an actual or predicted score change well above the threshold.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media (referred to herein as computer readable medium), such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine” may include at least one processor/controller, including one processor/controller to implement all of the instructions, at least two processors/controllers where one processor/controller operates on some of the instructions and other processor(s)/controller(s) operate on other instructions, or at least two processors/controllers where each processor/controller is capable of operating on the same instructions. Thus, for example, distributed systems or systems with shared resources are contemplated.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising using a processing system to perform a process that includes:
receiving lead placement information for a patient, the lead placement information being indicative of where at least one lead for a neurostimulator is implanted in the patient;
receiving clinical history for the patient, wherein the clinical history includes both first clinical effect data corresponding to times when the patient is not being treated with a therapy and second clinical effect data corresponding to times when the patient is being treated with the therapy;
employing at least one trained machine-learning model to provide predicted clinical effects, corresponding to untested stimulation parameter sets for the neurostimulator, based on the lead placement information, the first clinical effect data and the second clinical effect data; and
identifying a parameter set for use in delivering a therapy based at least in part on the predicted clinical effects for the untested stimulation parameter sets.

2. The method of claim 1, wherein the parameter set is identified using the predicted clinical effects to seed a search algorithm and implementing the search algorithm to determine the parameter set.

3. The method of claim 1, wherein the parameter set is identified using the predicted clinical effects to determine when to end searching for the parameter set.

4. The method of claim 1, wherein the parameter set is identified by the at least one trained machine-learning model based on the lead placement information, the first clinical effect data and the second clinical effect data.

5. The method of claim 1, further comprising using the at least one trained machine-learning model to suggest at least one of a lead entry point, a lead trajectory, a lead orientation, or a neural target based on the predicted clinical effects.

6. The method of claim 1, further comprising using the at least one trained machine-learning model to suggest, based on the predicted clinical effects, whether the patient is likely a responder or a non-responder to therapy.

7. The method of claim 1, wherein the at least one trained machine-learning model includes a regression model.

8. A method, comprising using a processing system to perform a process that includes:
accessing data stored in one or databases, wherein the data includes:
first clinical effect data corresponding to a first set of patient states for times when therapy is not delivered;
second clinical effect data corresponding to a second set of patient states for times when therapy is delivered; and
lead placement information for a neurostimulator that has a plurality of electrodes and that is configured to use at least one stimulation parameter set to deliver stimulation through at least some of the plurality of electrodes, the lead placement information being indicative of where at least one lead is implanted in the patient; and
providing machine learning to train at least one machine-learning model to predict a clinical effect for the neurostimulator programmed with untested stimulation parameter sets based on the lead placement information, the first clinical effect data, and the second clinical effect data.

9. The method of claim 8, wherein:
the data includes a plurality of tested stimulation sets and corresponding third clinical effect data;
each of the plurality of tested stimulation sets are used to deliver electrical stimulation through at least one lead implanted with a lead placement, and a corresponding instance of the third clinical effect data is determined when the electrical stimulation is delivered; and
the process further includes predicting, using the at least one machine-learning model, the clinical effect based further on the third clinical effect data and the plurality of tested stimulation sets.

10. The method of claim 9, wherein:
the plurality of tested stimulation sets includes at least one amplitude, at least one frequency, at least one pulse width for the electrical stimulation, active electrodes and at least one electrode fractionalization for distributing the electrical stimulation among the active electrodes, and
the process further includes predicting, using the at least one machine learning model, the clinical effect based further on one or more of the at least one amplitude, the at least one frequency, the at least one pulse width, the active electrodes or the at least one electrode fractionalization.

11. The method of claim 8, wherein the at least one machine-learning model includes a regression model.

12. The method of claim 8, wherein the lead placement information includes an implanted position of at least one deep brain stimulation (DBS) lead.

13. The method of claim 12, wherein the lead placement information includes information regarding at least one of a lead entry point, a lead trajectory, a lead orientation, or a neural target.

14. The method of claim 12, wherein the lead placement information includes relative lead position with respect to neuroanatomy, anatomical landmarks or anatomical coordinates defined by an anatomy of the patient or a stereotactic frame.

15. The method of claim 12, wherein the lead placement information includes information regarding an identifier of a medical facility or medical provider that implanted the at least one DBS lead.

16. A non-transitory machine-readable medium including instructions, which when executed by a machine, cause the machine to perform a method, comprising:
receiving lead placement information for a patient, the lead placement information being indicative of where at least one lead for a neurostimulator is implanted in the patient;
receiving clinical history for the patient, wherein the clinical history includes both first clinical effect data corresponding to times when the patient is not being treated with a therapy and second clinical effect data corresponding to times when the patient is being treated with the therapy;
employing at least one trained machine-learning model to provide predicted clinical effects, corresponding to untested stimulation parameter sets for the neurostimulator, based on the lead placement information, the first clinical effect data and the second clinical effect data; and
identifying a parameter set for use in delivering a therapy based at least in part on the predicted clinical effects for the untested stimulation parameter sets.

17. The non-transitory machine-readable medium of claim 16, wherein the parameter set is identified using the predicted clinical effects to seed a search algorithm and implementing the search algorithm to determine the parameter set.

18. The non-transitory machine-readable medium of claim 16, wherein the parameter set is identified using the predicted clinical effects to determine when to end searching for the parameter set.

19. The non-transitory machine-readable medium of claim 16, wherein the parameter set is identified by the at least one trained machine-learning model based on the lead placement information, the first clinical effect data and the second clinical effect data.

20. The non-transitory machine-readable medium of claim 16, further comprising using the at least one trained machine-learning model to suggest at least one of a lead entry point, a lead trajectory, a lead orientation, or a neural target based on the predicted clinical effects.
